SEQUENCE LISTING (54 sequences)

SEQ ID NO: 1 - protein, 904 aa, Homo sapiens
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 2 - DNA, 2715 bp, Homo sapiens
  source 1..2715: mol_type = genomic DNA
atgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaa

SEQ ID NO: 3 - protein, 839 aa, Homo sapiens
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 4 - protein, 23 aa, Homo sapiens
IGVSVLSVLVVSVVAVLVYKFYF

SEQ ID NO: 5 - protein, 183 aa, Homo sapiens
HLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 6 - DNA, 2520 bp, Homo sapiens
  source 1..2520: mol_type = genomic DNA
atgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 7 - protein, 1049 aa, Homo sapiens
MVFPMWTLKRQILILFNIILISKLLGARWFPKTLPCDVTLDVPKNHVIVDCTDKHLTEIPGGIPTNTTNLTLTINHIPDISPASFHRLDHLVEIDFRCNCVPIPLGSKNNMCIKRLQIKPRSFSGLTYLKSLYLDGNQLLEIPQGLPPSLQLLSLEANNIFSIRKENLTELANIEILYLGQNCYYRNPCYVSYSIEKDAFLNLTKLKVLSLKDNNVTAVPTVLPSTLTELYLYNNMIAKIQEDDFNNLNQLQILDLSGNCPRCYNAPFPCAPCKNNSPLQIPVNAFDALTELKVLRLHSNSLQHVPPRWFKNINKLQELDLSQNFLAKEIGDAKFLHFLPSLIQLDLSFNFELQVYRASMNLSQAFSSLKSLKILRIRGYVFKELKSFNLSPLHNLQNLEVLDLGTNFIKIANLSMFKQFKRLKVIDLSVNKISPSGDSSEVGFCSNARTSVESYEPQVLEQLHYFRYDKYARSCRFKNKEASFMSVNESCYKYGQTLDLSKNSIFFVKSSDFQHLSFLKCLNLSGNLISQTLNGSEFQPLAELRYLDFSNNRLDLLHSTAFEELHKLEVLDISSNSHYFQSEGITHMLNFTKNLKVLQKLMMNDNDISSSTSRTMESESLRTLEFRGNHLDVLWREGDNRYLQLFKNLLKLEELDISKNSLSFLPSGVFDGMPPNLKNLSLAKNGLKSFSWKKLQCLKNLETLDLSHNQLTTVPERLSNCSRSLKNLILKNNQIRSLTKYFLQDAFQLRYLDLSSNKIQMIQKTSFPENVLNNLKMLLLHHNRFLCTCDAVWFVWWVNHTEVTIPYLATDVTCVGPGAHKGQSVISLDLYTCELDLTNLILFSLSISVSLFLMVMMTASHLYFWDVWYIYHFCKAKIKGYQRLISPDCCYDAFIVYDTKDPAVTEWVLAELVAKLEDPREKHFNLCLEERDWLPGQPVLENLSQSIQLSKKTVFVMTDKYAKTENFKIAFYLSHQRLMDEKVDVIILIFLEKPFQKSKFLQLRKRLCGSSVLEWPTNPQAHPYFWQCLKNALATDNHVAYSQVFKETV

SEQ ID NO: 8 - DNA, 3150 bp, Homo sapiens
  source 1..3150: mol_type = genomic DNA
atggtgtttccaatgtggacactgaagagacaaattcttatcctttttaacataatcctaatttccaaactccttggggctagatggtttcctaaaactctgccctgtgatgtcactctggatgttccaaagaaccatgtgatcgtggactgcacagacaagcatttgacagaaattcctggaggtattcccacgaacaccacgaacctcaccctcaccattaaccacataccagacatctccccagcgtcctttcacagactggaccatctggtagagatcgatttcagatgcaactgtgtacctattccactggggtcaaaaaacaacatgtgcatcaagaggctgcagattaaacccagaagctttagtggactcacttatttaaaatccctttacctggatggaaaccagctactagagataccgcagggcctcccgcctagcttacagcttctcagccttgaggccaacaacatcttttccatcagaaaagagaatctaacagaactggccaacatagaaatactctacctgggccaaaactgttattatcgaaatccttgttatgtttcatattcaatagagaaagatgccttcctaaacttgacaaagttaaaagtgctctccctgaaagataacaatgtcacagccgtccctactgttttgccatctactttaacagaactatatctctacaacaacatgattgcaaaaatccaagaagatgattttaataacctcaaccaattacaaattcttgacctaagtggaaattgccctcgttgttataatgccccatttccttgtgcgccgtgtaaaaataattctcccctacagatccctgtaaatgcttttgatgcgctgacagaattaaaagttttacgtctacacagtaactctcttcagcatgtgcccccaagatggtttaagaacatcaacaaactccaggaactggatctgtcccaaaacttcttggccaaagaaattggggatgctaaatttctgcattttctccccagcctcatccaattggatctgtctttcaattttgaacttcaggtctatcgtgcatctatgaatctatcacaagcattttcttcactgaaaagcctgaaaattctgcggatcagaggatatgtctttaaagagttgaaaagctttaacctctcgccattacataatcttcaaaatcttgaagttcttgatcttggcactaactttataaaaattgctaacctcagcatgtttaaacaatttaaaagactgaaagtcatagatctttcagtgaataaaatatcaccttcaggagattcaagtgaagttggcttctgctcaaatgccagaacttctgtagaaagttatgaaccccaggtcctggaacaattacattatttcagatatgataagtatgcaaggagttgcagattcaaaaacaaagaggcttctttcatgtctgttaatgaaagctgctacaagtatgggcagaccttggatctaagtaaaaatagtatattttttgtcaagtcctctgattttcagcatctttctttcctcaaatgcctgaatctgtcaggaaatctcattagccaaactcttaatggcagtgaattccaacctttagcagagctgagatatttggacttctccaacaaccggcttgatttactccattcaacagcatttgaagagcttcacaaactggaagttctggatataagcagtaatagccattattttcaatcagaaggaattactcatatgctaaactttaccaagaacctaaaggttctgcagaaactgatgatgaacgacaatgacatctcttcctccaccagcaggaccatggagagtgagtctcttagaactctggaattcagaggaaatcacttagatgttttatggagagaaggtgataacagatacttacaattattcaagaatctgctaaaattagaggaattagacatctctaaaaattccctaagtttcttgccttctggagtttttgatggtatgcctccaaatctaaagaatctctctttggccaaaaatgggctcaaatctttcagttggaagaaactccagtgtctaaagaacctggaaactttggacctcagccacaaccaactgaccactgtccctgagagattatccaactgttccagaagcctcaagaatctgattcttaagaataatcaaatcaggagtctgacgaagtattttctacaagatgccttccagttgcgatatctggatctcagctcaaataaaatccagatgatccaaaagaccagcttcccagaaaatgtcctcaacaatctgaagatgttgcttttgcatcataatcggtttctgtgcacctgtgatgctgtgtggtttgtctggtgggttaaccatacggaggtgactattccttacctggccacagatgtgacttgtgtggggccaggagcacacaagggccaaagtgtgatctccctggatctgtacacctgtgagttagatctgactaacctgattctgttctcactttccatatctgtatctctctttctcatggtgatgatgacagcaagtcacctctatttctgggatgtgtggtatatttaccatttctgtaaggccaagataaaggggtatcagcgtctaatatcaccagactgttgctatgatgcttttattgtgtatgacactaaagacccagctgtgaccgagtgggttttggctgagctggtggccaaactggaagacccaagagagaaacattttaatttatgtctcgaggaaagggactggttaccagggcagccagttctggaaaacctttcccagagcatacagcttagcaaaaagacagtgtttgtgatgacagacaagtatgcaaagactgaaaattttaagatagcattttacttgtcccatcagaggctcatggatgaaaaagttgatgtgattatcttgatatttcttgagaagccctttcagaagtccaagttcctccagctccggaaaaggctctgtgggagttctgtccttgagtggccaacaaacccgcaagctcacccatacttctggcagtgtctaaagaacgccctggccacagacaatcatgtggcctatagtcaggtgttcaaggaaacggtctag

SEQ ID NO: 9 - protein, 1041 aa, Homo sapiens
MENMFLQSSMLTCIFLLISGSCELCAEENFSRSYPCDEKKQNDSVIAECSNRRLQEVPQTVGKYVTELDLSDNFITHITNESFQGLQNLTKINLNHNPNVQHQNGNPGIQSNGLNITDGAFLNLKNLRELLLEDNQLPQIPSGLPESLTELSLIQNNIYNITKEGISRLINLKNLYLAWNCYFNKVCEKTNIEDGVFETLTNLELLSLSFNSLSHVPPKLPSSLRKLFLSNTQIKYISEEDFKGLINLTLLDLSGNCPRCFNAPFPCVPCDGGASINIDRFAFQNLTQLRYLNLSSTSLRKINAAWFKNMPHLKVLDLEFNYLVGEIASGAFLTMLPRLEILDLSFNYIKGSYPQHINISRNFSKLLSLRALHLRGYVFQELREDDFQPLMQLPNLSTINLGINFIKQIDFKLFQNFSNLEIIYLSENRISPLVKDTRQSYANSSSFQRHIRKRRSTDFEFDPHSNFYHFTRPLIKPQCAAYGKALDLSLNSIFFIGPNQFENLPDIACLNLSANSNAQVLSGTEFSAIPHVKYLDLTNNRLDFDNASALTELSDLEVLDLSYNSHYFRIAGVTHHLEFIQNFTNLKVLNLSHNNIYTLTDKYNLESKSLVELVFSGNRLDILWNDDDNRYISIFKGLKNLTRLDLSLNRLKHIPNEAFLNLPASLTELHINDNMLKFFNWTLLQQFPRLELLDLRGNKLLFLTDSLSDFTSSLRTLLLSHNRISHLPSGFLSEVSSLKHLDLSSNLLKTINKSALETKTTTKLSMLELHGNPFECTCDIGDFRRWMDEHLNVKIPRLVDVICASPGDQRGKSIVSLELTTCVSDVTAVILFFFTFFITTMVMLAALAHHLFYWDVWFIYNVCLAKVKGYRSLSTSQTFYDAYISYDTKDASVTDWVINELRYHLEESRDKNVLLCLEERDWDPGLAIIDNLMQSINQSKKTVFVLTKKYAKSWNFKTAFYLALQRLMDENMDVIIFILLEPVLQHSQYLRLRQRICKSSILQWPDNPKAEGLFWQTLRNVVLTENDSRYNNMYVDSIKQY

SEQ ID NO: 10 - DNA, 3126 bp, Homo sapiens
  source 1..3126: mol_type = genomic DNA
atggaaaacatgttccttcagtcgtcaatgctgacctgcattttcctgctaatatctggttcctgtgagttatgcgccgaagaaaatttttctagaagctatccttgtgatgagaaaaagcaaaatgactcagttattgcagagtgcagcaatcgtcgactacaggaagttccccaaacggtgggcaaatatgtgacagaactagacctgtctgataatttcatcacacacataacgaatgaatcatttcaagggctgcaaaatctcactaaaataaatctaaaccacaaccccaatgtacagcaccagaacggaaatcccggtatacaatcaaatggcttgaatatcacagacggggcattcctcaacctaaaaaacctaagggagttactgcttgaagacaaccagttaccccaaataccctctggtttgccagagtctttgacagaacttagtctaattcaaaacaatatatacaacataactaaagagggcatttcaagacttataaacttgaaaaatctctatttggcctggaactgctattttaacaaagtttgcgagaaaactaacatagaagatggagtatttgaaacgctgacaaatttggagttgctatcactatctttcaattctctttcacacgtgccacccaaactgccaagctccctacgcaaactttttctgagcaacacccagatcaaatacattagtgaagaagatttcaagggattgataaatttaacattactagatttaagcgggaactgtccgaggtgcttcaatgccccatttccatgcgtgccttgtgatggtggtgcttcaattaatatagatcgttttgcttttcaaaacttgacccaacttcgatacctaaacctctctagcacttccctcaggaagattaatgctgcctggtttaaaaatatgcctcatctgaaggtgctggatcttgaattcaactatttagtgggagaaatagcctctggggcatttttaacgatgctgccccgcttagaaatacttgacttgtcttttaactatataaaggggagttatccacagcatattaatatttccagaaacttctctaaacttttgtctctacgggcattgcatttaagaggttatgtgttccaggaactcagagaagatgatttccagcccctgatgcagcttccaaacttatcgactatcaacttgggtattaattttattaagcaaatcgatttcaaacttttccaaaatttctccaatctggaaattatttacttgtcagaaaacagaatatcaccgttggtaaaagatacccggcagagttatgcaaatagttcctcttttcaacgtcatatccggaaacgacgctcaacagattttgagtttgacccacattcgaacttttatcatttcacccgtcctttaataaagccacaatgtgctgcttatggaaaagccttagatttaagcctcaacagtattttcttcattgggccaaaccaatttgaaaatcttcctgacattgcctgtttaaatctgtctgcaaatagcaatgctcaagtgttaagtggaactgaattttcagccattcctcatgtcaaatatttggatttgacaaacaatagactagactttgataatgctagtgctcttactgaattgtccgacttggaagttctagatctcagctataattcacactatttcagaatagcaggcgtaacacatcatctagaatttattcaaaatttcacaaatctaaaagttttaaacttgagccacaacaacatttatactttaacagataagtataacctggaaagcaagtccctggtagaattagttttcagtggcaatcgccttgacattttgtggaatgatgatgacaacaggtatatctccattttcaaaggtctcaagaatctgacacgtctggatttatcccttaataggctgaagcacatcccaaatgaagcattccttaatttgccagcgagtctcactgaactacatataaatgataatatgttaaagttttttaactggacattactccagcagtttcctcgtctcgagttgcttgacttacgtggaaacaaactactctttttaactgatagcctatctgactttacatcttcccttcggacactgctgctgagtcataacaggatttcccacctaccctctggctttctttctgaagtcagtagtctgaagcacctcgatttaagttccaatctgctaaaaacaatcaacaaatccgcacttgaaactaagaccaccaccaaattatctatgttggaactacacggaaacccctttgaatgcacctgtgacattggagatttccgaagatggatggatgaacatctgaatgtcaaaattcccagactggtagatgtcatttgtgccagtcctggggatcaaagagggaagagtattgtgagtctggagctaacaacttgtgtttcagatgtcactgcagtgatattatttttcttcacgttctttatcaccaccatggttatgttggctgccctggctcaccatttgttttactgggatgtttggtttatatataatgtgtgtttagctaaggtaaaaggctacaggtctctttccacatcccaaactttctatgatgcttacatttcttatgacaccaaagatgcctctgttactgactgggtgataaatgagctgcgctaccaccttgaagagagccgagacaaaaacgttctcctttgtctagaggagagggattgggacccgggattggccatcatcgacaacctcatgcagagcatcaaccaaagcaagaaaacagtatttgttttaaccaaaaaatatgcaaaaagctggaactttaaaacagctttttacttggctttgcagaggctaatggatgagaacatggatgtgattatatttatcctgctggagccagtgttacagcattctcagtatttgaggctacggcagcggatctgtaagagctccatcctccagtggcctgacaacccgaaggcagaaggcttgttttggcaaactctgagaaatgtggtcttgactgaaaatgattcacggtataacaatatgtatgtcgattccattaagcaatactaa

SEQ ID NO: 11 - protein, 1032 aa, Homo sapiens
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 12 - DNA, 3099 bp, Homo sapiens
  source 1..3099: mol_type = genomic DNA
atgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatag

SEQ ID NO: 13 - protein, 317 aa, Homo sapiens
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 14 - DNA, 954 bp, Homo sapiens
  source 1..954: mol_type = genomic DNA
atgactatggagacccaaatgtctcagaatgtatgtcccagaaacctgtggctgcttcaaccattgacagttttgctgctgctggcttctgcagacagtcaagctgcagctcccccaaaggctgtgctgaaacttgagcccccgtggatcaacgtgctccaggaggactctgtgactctgacatgccagggggctcgcagccctgagagcgactccattcagtggttccacaatgggaatctcattcccacccacacgcagcccagctacaggttcaaggccaacaacaatgacagcggggagtacacgtgccagactggccagaccagcctcagcgaccctgtgcatctgactgtgctttccgaatggctggtgctccagacccctcacctggagttccaggagggagaaaccatcatgctgaggtgccacagctggaaggacaagcctctggtcaaggtcacattcttccagaatggaaaatcccagaaattctcccatttggatcccaccttctccatcccacaagcaaaccacagtcacagtggtgattaccactgcacaggaaacataggctacacgctgttctcatccaagcctgtgaccatcactgtccaagtgcccagcatgggcagctcttcaccaatggggatcattgtggctgtggtcattgcgactgctgtagcagccattgttgctgctgtagtggccttgatctactgcaggaaaaagcggatttcagccaattccactgatcctgtgaaggctgcccaatttgagccacctggacgtcaaatgattgccatcagaaagagacaacttgaagaaaccaacaatgactatgaaacagctgacggcggctacatgactctgaaccccagggcacctactgacgatgataaaaacatctacctgactcttcctcccaacgaccatgtcaacagtaataactaa

SEQ ID NO: 15 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 16 - DNA, 765 bp, Homo sapiens
  source 1..765: mol_type = genomic DNA
atgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatga

SEQ ID NO: 17 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 18 - DNA, 261 bp, Homo sapiens
  source 1..261: mol_type = genomic DNA
atgattccagcagtggtcttgctcttactccttttggttgaacaagcagcggccctgggagagcctcagctctgctatatcctggatgccatcctgtttctgtatggaattgtcctcaccctcctctactgtcgactgaagatccaagtgcgaaaggcagctataaccagctatgagaaatcagatggtgtttacacgggcctgagcaccaggaaccaggagacttacgagactctgaagcatgagaaaccaccacagtag

SEQ ID NO: 19 - protein, 296 aa, Homo sapiens
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 20 - DNA, 954 bp, Homo sapiens
  source 1..954: mol_type = genomic DNA
atgcgacccgaccgcgctgaggctccaggaccgcccgccatggctgcaggaggtcccggcgcggggtctgcggccccggtctcctccacatcctcccttcccctggctgctctcaacatgcgagtgcggcgccgcctgtctctgttcttgaacgtgcggacacaggtggcggccgactggaccgcgctggcggaggagatggactttgagtacttggagatccggcaactggagacacaagcggaccccactggcaggctgctggacgcctggcagggacgccctggcgcctctgtaggccgactgctcgagctgcttaccaagctgggccgcgacgacgtgctgctggagctgggacccagcattgaggaggattgccaaaagtatatcttgaagcagcagcaggaggaggctgagaagcctttacaggtggccgctgtagacagcagtgtcccacggacagcagagctggcgggcatcaccacacttgatgaccccctggggcatatgcctgagcgtttcgatgccttcatctgctattgccccagcgacatccagtttgtgcaggagatgatccggcaactggaacagacaaactatcgactgaagttgtgtgtgtctgaccgcgatgtcctgcctggcacctgtgtctggtctattgctagtgagctcatcgaaaagaggttggctagaaggccacggggtgggtgccgccggatggtggtggttgtctctgatgattacctgcagagcaaggaatgtgacttccagaccaaatttgcactcagcctctctccaggtgcccatcagaagcgactgatccccatcaagtacaaggcaatgaagaaagagttccccagcatcctgaggttcatcactgtctgcgactacaccaacccctgcaccaaatcttggttctggactcgccttgccaaggccttgtccctgccctga

SEQ ID NO: 21 - protein, 153 aa, Homo sapiens
MAPAAATGGSTLPSGFSVFTTLPDLLFIFEFIFGGLVWILVASSLVPWPLVQGWVMFVSVFCFVATTTLIILYIIGAHGGETSWVTLDAAYHCTAALFYLSASVLEALATITMQDGFTYRHYHENIAAVVFSYIATLLYVVHAVFSLIRWKSS

SEQ ID NO: 22 - DNA, 462 bp, Homo sapiens
  source 1..462: mol_type = genomic DNA
atggcccccgcagcggcgacggggggcagcaccctgcccagtggcttctcggtcttcaccaccttgcccgacttgctcttcatctttgagtttatcttcgggggcctggtgtggatcctggtggcctcctccctggtgccctggcccctggtccagggctgggtgatgttcgtgtctgtgttctgcttcgtggccaccaccaccttgatcatcctgtacataattggagcccacggtggagagacttcctgggtcaccttggacgcagcctaccactgcaccgctgccctcttttacctcagcgcctcagtcctggaggccctggccaccatcacgatgcaagacggcttcacctacaggcactaccatgaaaacattgctgccgtggtgttctcctacatagccactctgctctacgtggtccatgcggtgttctctttaatcagatggaagtcttcataa

SEQ ID NO: 23 - protein, 712 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 24 - DNA, 2139 bp, Homo sapiens
  source 1..2139: mol_type = genomic DNA
atggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctga

SEQ ID NO: 25 - protein, 257 aa, synthetic construct
  REGION 1..257: note = ScFv fragment
MSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDL

SEQ ID NO: 26 - protein, 5 aa, synthetic construct
  REGION 1..5: note = Short hinge linker
MNKTI

SEQ ID NO: 27 - protein, 55 aa, synthetic construct
  REGION 1..55: note = Long hinge linker
NDFACTCEHQSFLQWIKDQRQLLVEVERMECATPSDKQGMPVLSLNITCQMNKTI

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 12 aa, Homo sapiens
ESKYGPPCPPCP

SEQ ID NO: 30 - protein, 119 aa, Homo sapiens
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 31 - protein, 228 aa, Homo sapiens
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 32 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 33 - protein, 68 aa, synthetic construct
  REGION 1..68: note = Mutated CD8 hinge
SALSNSIMYFSHFVPVFLPAKPTTTPAPRPPTPAPTIASQPLSLRPEASRPAAGGAVHTRGLDFASDI

SEQ ID NO: 34 - protein, 15 aa, synthetic construct
  REGION 1..15: note = GS linker
GGGGSGGGGSGGGGS

SEQ ID NO: 35 - protein, 496 aa, synthetic construct
  REGION 1..496: note = Chimeric antigen receptor
  SIGNAL 1..18
  REGION 19..275: note = MISC_FEATURE - TK1-ScFv
  REGION 276..290: note = MISC_FEATURE - GS-Linker
  REGION 291..313: note = MISC_FEATURE - TLR4-Transmembrane domain
  REGION 314..496: note = MISC_FEATURE - TLR4-Cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 36 - protein, 500 aa, synthetic construct
  REGION 1..500: note = Chimeric antigen receptor
  SIGNAL 1..18
  REGION 19..275: note = MISC_FEATURE - TK1-ScFv
  REGION 276..290: note = MISC_FEATURE - GS-Linker
  REGION 291..295: note = MISC_FEATURE - LRR Short Hinge
  REGION 296..318: note = MISC_FEATURE - TLR4 transmembrane domain
  REGION 319..500: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATS

SEQ ID NO: 37 - protein, 551 aa, synthetic construct
  REGION 1..551: note = Chimeric antigen receptor
  SIGNAL 1..18: note = Singal peptide
  REGION 19..275: note = MISC_FEATURE - TK1-ScFv
  REGION 269..551: note = MISC_FEATURE - TLR4 cytosolic domain
  REGION 276..290: note = MISC_FEATURE - GS-Linker
  REGION 291..345: note = MISC_FEATURE - LRR long hinge
  REGION 346..368: note = MISC_FEATURE - TLR4 transmembrane domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 38 - protein, 508 aa, synthetic construct
  REGION 1..508: note = Chimeric antigen receptor
  SIGNAL 1..18
  REGION 19..275: note = MISC_FEATURE - TK1 ScFv
  REGION 276..290: note = MISC_FEATURE - GS linker
  REGION 291..302: note = MISC_FEATURE - IgG4 short hinge
  REGION 303..325: note = MISC_FEATURE - TLR4 transmembrane domain
  REGION 326..508: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 39 - protein, 615 aa, synthetic construct
  REGION 1..615: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..409: note = MISC_FEATURE - IgG4 medium hinge
  REGION 410..432: note = MISC_FEATURE - TLR4 transmembrane domain
  REGION 433..615: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 40 - protein, 724 aa, synthetic construct
  REGION 1..724: note = Chimeric antigen receptor
  SIGNAL 1..18
  REGION 19..275: note = MISC_FEATURE - TK1 ScFv
  REGION 276..290: note = MISC_FEATURE - GS linker
  REGION 291..518: note = MISC_FEATURE - IgG4 long hinge
  REGION 519..541: note = MISC_FEATURE - TLR4 transmembrane domain
  REGION 542..724: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 41 - protein, 564 aa, synthetic construct
  REGION 1..564: note = Chimeric antigen receptor
  SIGNAL 1..18: note = Signal peptide
  REGION 19..275: note = MISC_FEATURE - anti-TK1 ScFv
  REGION 276..290: note = MISC_FEATURE - GS linkder
  REGION 291..358: note = MISC_FEATURE - Mutated CD8 hinge
  REGION 359..381: note = MISC_FEATURE - TLR4 transmembrane domain
  REGION 382..564: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 42 - protein, 564 aa, synthetic construct
  REGION 1..564: note = Chimeric antigen receptor
  SIGNAL 1..18: note = Sginal peptide
  REGION 19..275: note = MISC_FEATURE - TK1 ScFv
  REGION 276..290: note = MISC_FEATURE - GS linker
  REGION 291..358: note = MISC_FEATURE - GS linker
  REGION 359..381: note = MISC_FEATURE - TLR4 transmembrane domain
  REGION 382..564: note = MISC_FEATURE - TLR4 cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 43 - protein, 378 aa, synthetic construct
  REGION 1..378: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..311: note = MISC_FEATURE - FCGR3A transmembrane domain
  REGION 312..336: note = MISC_FEATURE - FCGR3A cytosolic domain
  REGION 337..378: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 44 - protein, 446 aa, synthetic construct
  REGION 1..446: note = Chimeric antigen receptor
  SIGNAL 1..18: note = Signal peptide
  REGION 19..275: note = MISC_FEATURE - anti-TK1 ScFv
  REGION 276..290: note = MISC_FEATURE - GS linker
  REGION 291..358: note = MISC_FEATURE - Modified CD8a linker
  REGION 359..379: note = MISC_FEATURE - FCGR3A transmembrane domain
  REGION 380..404: note = MISC_FEATURE - FCGR3A cytosolic domain
  REGION 405..446: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 45 - protein, 446 aa, synthetic construct
  REGION 1..446: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..358: note = MISC_FEATURE - Portion of CD8 hinge
  REGION 359..379: note = MISC_FEATURE - FCGR3A transmembrane domain
  REGION 380..404: note = MISC_FEATURE - FCGR3A cytosolic domain
  REGION 405..446: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 46 - protein, 391 aa, synthetic construct
  REGION 1..391: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..303: note = MISC_FEATURE - IgG4 short hinge
  REGION 304..324: note = MISC_FEATURE - FCGR3A transmembrane domain
  REGION 325..349: note = MISC_FEATURE - FCGR3A cytosolic domain
  REGION 350..391: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 47 - protein, 497 aa, synthetic construct
  REGION 1..497: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linkder
  REGION 291..409: note = MISC_FEATURE - IgG4 119 aa hinge
  REGION 410..430: note = MISC_FEATURE - FCGR3A transmembrane domain
  REGION 431..455: note = MISC_FEATURE - FCGR3A cytosolic domain
  REGION 456..497: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 48 - protein, 607 aa, synthetic construct
  REGION 1..607: note = Chimeric angtigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..519: note = MISC_FEATURE - IgG4 long hinge
  REGION 520..540: note = MISC_FEATURE - FCGR3A transmembrane domain
  REGION 541..565: note = MISC_FEATURE - FCGR3A cytosolic domain
  REGION 566..607: note = MISC_FEATURE - FCER1G cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIVSFCLVMVLLFAVDTGLYFSVKTNIRSSTRDWKDHKFKWRKDPQDKRLKIQVRKAAITSYEKSDGVYTGLSTRNQETYETLKHEKPPQ

SEQ ID NO: 49 - protein, 390 aa, synthetic construct
  REGION 1..390: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..312: note = MISC_FEATURE - FCGR2A transmembrane domain
  REGION 313..390: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 50 - protein, 458 aa, synthetic construct
  REGION 1..458: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..358: note = MISC_FEATURE - Modified CD8a hinge
  REGION 359..380: note = MISC_FEATURE - FCGR2A transmembrane domain
  REGION 381..458: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEASRPAAGGAVHTRGLDFASDIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 51 - protein, 458 aa, synthetic construct
  REGION 1..458: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..358: note = MISC_FEATURE - Portion of CD8a linker
  REGION 359..380: note = MISC_FEATURE - FCGR2A transmembrane domain
  REGION 381..458: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSSALSNSIMYFSHFVPVFLPAKPTTTPAPRPPTPAPTIASQPLSLRPEACRPAAGGAVHTRGLDFACDIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 52 - protein, 403 aa, synthetic construct
  REGION 1..403: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..303: note = MISC_FEATURE - IgG4 short hinge
  REGION 304..325: note = MISC_FEATURE - FCGR2A transmembrane domain
  REGION 326..403: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 53 - protein, 509 aa, synthetic construct
  REGION 1..509: note = Chimeric antigen receptor
  SIGNAL 1..18: note = Signal peptide
  REGION 19..275: note = MISC_FEATURE - TK1 ScFv
  REGION 276..290: note = MISC_FEATURE - GS linker
  REGION 291..409: note = MISC_FEATURE - IgG4 119 aa hinge
  REGION 410..431: note = MISC_FEATURE - FCGR2A transmembrane domain
  REGION 432..509: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 54 - protein, 619 aa, synthetic construct
  REGION 1..619: note = Chimeric antigen receptor
  SIGNAL 1..18: note = Signal peptide
  REGION 19..275: note = MISC_FEATURE - TK! ScFv
  REGION 276..290: note = MISC_FEATURE - GS linker
  REGION 291..519: note = MISC_FEATURE - IgG4 long hinge
  REGION 520..541: note = MISC_FEATURE - FCGR2A transmembrane domain
  REGION 542..619: note = MISC_FEATURE - FCGR2A cytosolic domain
MDFQVQIISFLLISASVIMSRGQIVLSQSPAILSASPGEKVTMTCRASSSVSYMHFYQQKPGSSPKPWIYATSNLASGVPARFSGSGSGTSFSLTISRVEAEDAATYYCQQWSSNPPTFGSGTKLEIKSGGGGSGGGGSGGGGSMAVVTGVNSEVQLQQSGAELVKPGASVKLSCTASGFNIKDTYIHWVKQRPEQGLEWIGRIDPANGNTKYDPKFQGKATITTDTSFNTAYLQLSSLTSEDTAVYYCAKVGYGHWYFDVWGAGTTVTVSSVDLGGGGSGGGGSGGGGSESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKIIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

(19) United States
(12) Patent Application Publication
(10) Pub. No.: US 2025/0075181 A1
(43) Pub. Date: Mar. 6, 2025

(54) Transgenic Macrophages, Chimeric Antigen Receptors, and Associated Methods

(71) Applicant: Thunder Biotech, Inc., Provo, UT (US)

(72) Inventor: Kim Leslie O'Neill, Provo, UT (US)

(21) Appl. No.: 18/731,958

(22) Filed: Jun. 3, 2024

Related U.S. Application Data
(63) Continuation of application No. 17/146,024, filed on Jan. 11, 2021, now Pat. No. 12,024,719.
Application No. 17/146,024 is a continuation of application No. 16/572,368, filed on Sep. 16, 2019, now Pat. No. 10,889,803.
Application No. 16/572,368 is a continuation of application No. 15/597,822, filed on May 17, 2017, now Pat. No. 10,415,017.

(51) Int. Cl. C12N 5/0786; A61K 39/00; C07K 14/705; C07K 16/30; C07K 16/40

(52) U.S. Cl. CPC C12N 5/0645; A61K 40/17; A61K 40/24; A61K 40/31; A61K 40/4251; C07K 14/705; C07K 16/30; C07K 16/40; C12Y 207/01021; A61K 2239/55; C07K 2317/622; C07K 2319/30; C07K 2319/32; C07K 2319/33; C07K 2319/70; C12N 2510/00

(57) ABSTRACT

Described herein are chimeric receptors. Chimeric receptors comprise a cytoplasmic domain; a transmembrane domain; and an extracellular domain. In embodiments, the cytoplasmic domain comprises a cytoplasmic portion of a receptor that when activated polarizes a macrophage. In further embodiments, a wild-type protein comprising the cytoplasmic portion does not comprise the extracellular domain of the chimeric receptor. In embodiments, the binding of a ligand to the extracellular domain of the chimeric receptor activates the intracellular portion of the chimeric receptor. Activation of the intracellular portion of the chimeric receptor may polarize the macrophage into an M1 or M2 macrophage.

### CROSS-REFERENCE TO RELATED APPLICATION

This application is a continuation of U.S. patent application Ser. No. 17/146,024, filed Jan. 11, 2021, pending, which is a continuation of U.S. patent application Ser. No. 16/572,368, filed Sep. 16, 2019, issued as U.S. Pat. No. 11,999,970, which is a continuation of and claims priority to U.S. patent application Ser. No. 15/597,822, filed May 17, 2017, now issued as U.S. Pat. No. 10,415,017, the entireties of which are hereby incorporated by reference.

### INCORPORATION BY REFERENCE

The ST.26 XML Sequence listing named “2024 Jun. 3-Sequence listing ST26”, created on Jun. 3, 2024, and having a size of 142,587 bytes, is hereby incorporated herein by this reference in its entirety.

### TECHNICAL FIELD

The present disclosure relates generally to biotechnology. More specifically, the present disclosure relates to chimeric antigen receptors, nucleic acids encoding chimeric antigen receptors, macrophages harboring chimeric antigen receptors and/or nucleic acids encoding, and associated methods.

### BACKGROUND

Cancer consists of a group of diseases which involve unregulated cell growth and death, genome instability and mutations, tumor-promoting inflammation, induction of angiogenesis, immune system evasion, deregulation of metabolic pathways, immortal cell replication, and metastatic tissue invasion [1]. Cancer is the second leading cause of death in the United States after heart disease [2]. More than 1.6 million new cases of cancer are projected to be diagnosed each year, with more than 580,000 Americans expected to die (about 1600 cancer deaths per day), accounting for nearly 1 in 4 of all American deaths [2,3].

The immune system plays an important role in the development and progression of cancer. Immune cell infiltration to the tumor site can adversely affect malignancy progression and metastasis [4, 5]. Infiltration of macrophages into the tumor site has been shown to account for more than 50% of the tumor mass in certain breast cancer cases suggesting macrophages have a significant role in tumor progression [6-8].

Macrophages are cells derived from the myeloid lineage and belong to the innate immune system. They are derived from blood monocytes that migrate into tissue. One of their main functions is to phagocytose microbes and clear cellular debris. They also play an important role in both the initiation and resolution of inflammation [9, 10]. Moreover, macrophages can display different responses, ranging from pro-inflammatory to anti-inflammatory, depending on

the type of stimuli they receive from the surrounding microenvironment [11]. Two major macrophage phenotypes have been proposed which correlate with extreme macrophage responses: M1 and M2.

M1 pro-inflammatory macrophages are activated upon contact with certain molecules such as lipopolysaccharide (LPS), IFN-γ, IL-1β, TNF-α, and Toll-like receptor engagement. M1 macrophages constitute a potent arm of the immune system deployed to fight infections. They are capable of either direct (pathogen pattern recognition receptors) or indirect (Fc receptors, complement receptors) recognition of the pathogen. They are also armed in their ability to produce reactive oxygen species (ROS) as means to help killing pathogens. In addition, MI macrophages secrete pro-inflammatory cytokines and chemokines attracting other types of immune cells and integrating/orchestrating the immune response. M1 activation is induced by IFN-g, TNFa, GM-CSF, LPS and other toll-like receptors (TLR) ligands.

In contrast, M2 anti-inflammatory macrophages, also known as alternatively activated macrophages, are activated by anti-inflammatory molecules such as IL-4, IL-13, and IL-10 [12, 13]. M2 macrophages exhibit immunomodulatory, tissue repair, and angiogenesis properties which allow them to recruit regulatory T cells to sites of inflammation. M2 macrophages do not constitute a uniform population and often are further subdivided into M2a, M2b and M2c categories. The common denominator of all three subpopulations is high IL-10 production accompanied by low production of IL-12. One of their signatures is production of enzyme Arginase-1 that depletes L-arginine thereby suppressing T cell responses and depriving iNOS of its substrate.

The in vivo molecular mechanisms of macrophage polarization are poorly characterized because of the variety of signals macrophages experience in the cellular microenvironment [10, 14]. In recent years, progress has been made in identifying in vivo macrophage polarization under physiological conditions such as ontogenesis, pregnancy, and pathological conditions such as allergies, chronic inflammation, and cancer. We do know, however, that in vitro macrophage polarization is plastic and macrophages, with the help of cytokines, can be polarized back and forth to either phenotype [15, 16]. Interferon gamma (IFN-γ) and IL-4 are two cytokines that can polarize macrophages to M1 and M2 phenotypes, respectively [15].

The presence of macrophages is crucial for tumor progression and growth, and has implications in determining prognosis [17, 18]. Because macrophages can exhibit both pro-inflammatory and anti-inflammatory properties, it is important to understand their polarization and function in tumor progression and metastasis.

### Macrophage Polarization

The tumor microenvironment can affect macrophage polarization. The process of polarization can be diverse and complex because of the hostile environment of IL-10, glucocorticoid hormones, apoptotic cells, and immune complexes that can interfere with innate immune cells function [11, 19]. The mechanisms of polarization are still unclear but we know they involve transcriptional regulation. For example, macrophages exposed to LPS or IFN-γ will polarize towards an M1 phenotype, whereas macrophages exposed to IL-4 or IL-13 will polarize towards an M2 phenotype. LPS or IFN-γ can interact with Toll-like receptor 4 (TLR4) on the surface of macrophages inducing the Trif and MyD88 pathways, inducing the activation of transcription factors IRF3, AP-1, and NFKB and thus activating TNFs genes, interferon genes, CXCL10, NOS2, IL-12, etc., which are necessary in a pro-inflammatory M1 macrophage response [20]. Similarly, IL-4 and IL-13 bind to IL-4R, activation the Jak/Stat6 pathway, which regulates the expression of CCL17, ARG1, IRF4, IL-10, SOCS3, etc., which are genes associated with an anti-inflammatory response (M2 response).

Additional mechanisms of macrophage polarization include microRNA (miRNA) micromanagement. miRNAs are small non-coding RNA of 22 nucleotides in length that regulate gene expression post-transcriptionally, as they affect the rate of mRNA degradation. Several miRNAs have been shown to be highly expressed in polarized macrophages, especially miRNA-155, miRNA-125, miRNA-378 (MI polarization), and miRNA let-7c, miRNA-9, miRNA-21, miRNA-146, miRNA147, miRNA-187 (M2 polarization) [21].

Macrophage polarization is a complex process, were macrophages behave and elicit different responses depending on microenvironment stimuli. Therefore, macrophage polarization is better represented by a continuum of activation states where M1 and M2 phenotypes are the extremes of the spectrum. In recent years, there has been much controversy on the definition/description of macrophage activation and macrophage polarization. A recent paper published by Murray et al., in which they describe a set of standards to be considered for the consensus definition/description of macrophage activation, polarization, activators, and markers. This publication was much needed for the definition and characterization of activated/polarized macrophages [22].

### M1 Phenotype

M1 pro-inflammatory macrophages or classically activated macrophages are aggressive, highly phagocytic, and produce large amounts of reactive oxygen and nitrogen species, thereby promoting a Th1 response [11]. MI macrophages secrete high levels of two important inflammatory cytokines, IL-12 and IL-23. IL-12 induces the activation and clonal expansion of Th17 cells, which secrete high amounts of IL-17, which contributes to inflammation [23]. These characteristics allow M1 macrophages to control metastasis, suppress tumor growth, and control microbial infections [24]. Moreover, the infiltration and recruitment of M1 macrophages to tumor sites correlates with a better prognosis and higher overall survival rates in patients with solid tumors [17, 18, 25-28].

Polarization of macrophages to the M1 phenotype is regulated in vitro by inflammatory signals such as IFN-γ, TNF-α, IL-1β and LPS as well as transcription factors and miRNAs [29, 30]. Classically activated macrophages initiate the induction of the STATI transcription factor which targets CXCL9, CXCL10 (also known as IP-10), IFN regulatory factor-1, and suppressor of cytokine signaling-1 [31]. Cytokine signaling-1 protein functions downstream of cytokine receptors, and takes part in a negative feedback loop to attenuate cytokine signaling. In the tumor microenvironment, Notch signaling plays an important role in the polarization of M1 macrophages, as it allows transcription factor RBP-J to regulate classical activation. Macrophages that are deficient in Notch signaling express an M2 phenotype regardless of other extrinsic inducers [32]. One crucial miRNA, miRNA-155, is upregulated when macrophages are transitioning from M2 to M1; M1 macrophages overexpressing miRNA-155 are generally more aggressive and are associated with tumor reduction [33]. Moreover, miRNA-342-5p has been found to foster a greater inflammatory response in macrophages by targeting Akt1 in mice. This miRNA also promotes the upregulation of Nos2 and IL-6, both of which act as inflammatory signals for macrophages [34]. Other miRNAs such as miRNA-125 and miRNA-378 have also been shown to be involved in the classical activation pathway of macrophages (M1) [35].

Classically activated macrophages are thought to play an important role in the recognition and destruction of cancer cells as their presence usually indicates good prognosis. After recognition, malignant cells can be destroyed by MI macrophages through several mechanisms, which include contact-dependent phagocytosis and cytotoxicity (i.e., cytokine release such as TNF-α) [24]. Environmental signals such as the tumor microenvironment or tissue-resident cells, however, can polarize M1 macrophages to M2 macrophages. In vivo studies of murine macrophages have shown that macrophages are plastic in their cytokine and surface marker expression and that re-polarizing macrophages to an M1 phenotype in the presence of cancer can help the immune system reject tumors [19].

### M2 Phenotype

M2 macrophages are anti-inflammatory and aid in the process of angiogenesis and tissue repair. They express scavenger receptors and produce large quantities of IL-10 and other anti-inflammatory cytokines [33, 36]. Expression of IL-10 by M2 macrophages promotes a Th2 response. Th2 cells consequently upregulate the production of IL-3 and IL-4. IL-3 stimulates proliferation of all cells in the myeloid lineage (granulocytes, monocytes, and dendritic cells), in conjunction with other cytokines, e.g., Erythropoietin (EPO), Granulocyte macrophage colony-stimulating factor (GM-CSF), and IL-6. IL-4 is an important cytokine in the healing process because it contributes to the production of the extracellular matrix [23]. M2 macrophages exhibit functions that may help tumor progression by allowing blood vessels to feed the malignant cells and thus promoting their growth. The presence of macrophages (thought to be M2) in the majority of solid tumors negatively correlates with treatment success and longer survival rates [37]. Additionally, the presence of M2 macrophages has been linked to the metastatic potential in breast cancer. Lin and colleagues found that carly recruitment of macrophages to the breast tumor sites in mice increase angiogenesis and incidence of malignancy [38]. It is thought that the tumor microenvironment helps macrophages maintain an M2 phenotype [23, 39]. Anti-inflammatory signals present in the tumor microenvironment such as adiponectin and IL-10 can enhance an M2 response [41].

### Tumor-Associated Macrophages (TAMs)

Cells exposed to a tumor microenvironment behave differently. For example, tumor-associated macrophages found in the periphery of solid tumors are thought to help promote tumor growth and metastasis, and have an M2-like phenotype [42]. Tumor-associated macrophages can be either tissue resident macrophages or recruited macrophages derived from the bone marrow (macrophages that differentiate from monocytes to macrophages and migrate into tissue). A study by Cortez-Retamozo found that high numbers of TAM precursors in the spleen migrate to the tumor stroma, suggesting this organ as a TAM reservoir also [43]. TAM precursors found in the spleen were found to initiate migration through their CCR2 chemokine receptor [43]. Recent studies have found CSF-1 as the primary factor that attracts macrophages to the tumor periphery, and that CSF-1 production by cancer cells predicts lower survival rates and it indicates an overall poor prognosis [44-46]. Other cytokines such as TNF-α and IL-6 have been also linked to the accumulation/recruitment of macrophages to the tumor periphery [45].

It is thought that macrophages that are recruited around the tumor borders are regulated by an “angiogenic switch” that is activated in the tumor. The angiogenic switch is defined as the process by which the tumor develops a high density network of blood vessels that potentially allows the tumor to become metastatic, and is necessary for malignant transition. In a breast cancer mouse model, it was observed that the presence of macrophages was required for a full angiogenic switch. When macrophage maturation, migration, and accumulation around the tumor was delayed, the angiogenic switch was also delayed suggesting that the angiogenic switch does not occur in the absence of macrophages and that macrophage presence is necessary for malignancy progression [47]. Moreover, the tumor stromal cells produce chemokines such as CSF1, CCL2, CCL3, CCL5, and placental growth factor that will recruit macrophages to the tumor surroundings. These chemokines provide an environment for macrophages to activate the angiogenic switch, in which macrophages will produce high levels of IL-10, TGF-β, ARG-1 and low levels of IL-12, TNF-α, and IL-6. The level of expression of these cytokines suggests macrophages modulate immune evasion. It is important to note that macrophages are attracted to hypoxic tumor environments and will respond by producing hypoxia-inducible factor-1α (HIF-1α) and HIF-2α, which regulate the transcription of genes associated with angiogenesis. During the angiogenic switch, macrophages can also secrete VEGF (stimulated by the NF-κB pathway), which will promote blood vessel maturation and vascular permeability [48].

Tumor-associated macrophages are thought to be able to maintain their M2-like phenotype by receiving polarization signals from malignant cells such as IL-IR and MyD88, which are mediated through IkB kinase β and NF-KB signaling cascade. Inhibition of NF-KB in

TAMs promotes classical activation [40]. Moreover, another study suggested that p50 NF-KB subunit was involved in suppression of M1 macrophages, and reduction of inflammation promoted tumor growth. A p50 NF-KB knock-out mouse generated by Saccani et al. suggested that M1 aggressiveness was restored upon p50 NF-KB knockout, reducing tumor survival [49].

Because the tumor mass contains a great number of M2-like macrophages, TAMs can be used as a target for cancer treatment. Reducing the number of TAMs or polarizing them towards an M1 phenotype can help destroy cancer cells and impair tumor growth [50-52]. Luo and colleagues used a vaccine against legumain, a cysteine protease and stress protein upregulated

in TAMs thought to be a potential tumor target [52]. When the vaccine against legumain was administered to mice, genes controlling angiogenesis were downregulated and tumor growth was halted [52].

### Metabolism and Activation Pathways

Metabolic alterations present in tumor cells are controlled by the same genetic mutations that produce cancer [53]. As a result of these metabolic alterations, cancer cells are able to produce signals that can modify the polarization of macrophages and promote tumor growth [54, 55].

M1 and M2 macrophages demonstrate distinct metabolic patterns that reflect their dissimilar behaviors [56]. The M1 phenotype increases glycolysis and skews glucose metabolism towards the oxidative pentose phosphate pathway, thereby decreasing oxygen consumption and consequently producing large amounts of radical oxygen and nitrogen species as well as inflammatory cytokines such as TNF-α, IL-12, and IL-6 [56, 57]. The M2 phenotype increases fatty acid intake and oxidation, which decreases flux towards the pentose phosphate pathway while increasing the overall cell redox potential, consequently upregulating scavenger receptors and immunomodulatory cytokines such as IL-10 and TGF-B [56].

Multiple metabolic pathways play important roles in macrophage polarization. Protein kinases, such as Akt1 and Akt2, alter macrophage polarization by allowing cancer cells to survive, proliferate, and use an intermediary metabolism [58]. Other protein kinases can direct macrophage polarization through glucose metabolism by increasing glycolysis and decreasing oxygen consumption [57, 59]. Shu and colleagues were the first to visualize macrophage metabolism and immune response in vivo using a PET scan and a glucose analog [60].

L-arginine metabolism also exhibits discrete shifts important to cytokine expression in macrophages and exemplifies distinct metabolic pathways which alter TAM-tumor cell interactions [61]. Classically activated (M1) macrophages favor inducible nitric oxide synthase (iNOS). The iNOS pathway produces cytotoxic nitric oxide (NO), and consequently exhibits anti-tumor behavior. Alternatively activated (M2) macrophages have been shown to favor the arginase pathway and produce ureum and 1-ornithine, which contribute to progressive tumor cell growth [61, 62].

Direct manipulation of metabolic pathways can alter macrophage polarization. The carbohydrate kinase-like protein (CARKL) protein, which plays a role in glucose metabolism, has been used to alter macrophage cytokine signatures [56, 57]. When CARKL is knocked down by RNAi, macrophages tend to adopt an M1-like metabolic pathway (metabolism skewed towards glycolysis and decreased oxygen consumption). When CARKL is overexpressed, macrophages adopt an M2-like metabolism (decreased glycolytic flux and more oxygen consumption) [56].When macrophages adopt an M1-like metabolic state through LPS/TLR4 engagement, CARKL levels decrease, genes controlled by the NFKB pathway are activated (TNF-α, IL-12, and IL-6), and cell redox potential increases due to growing concentrations of NADH: NAD+ and GSH: GSSSG complexes. During an M2-like metabolic state, macrophages upregulate CARKL and genes regulated by STAT6/IL-4 (IL-10 and TGF-β).

Obesity can also affect macrophage polarization. Obesity is associated with a state of chronic inflammation, an environment that drives the IL4/STAT6 pathway to activate NKT cells, which drive macrophages towards an M2 response. During late-stage diet-induced obesity, macrophages migrate to adipose tissue, where immune cells alter levels of TH1 or TH2 cytokine expression in the adipose tissue, causing an M2 phenotype bias and possibly increased insulin sensitivity [63].

M1 phenotype bias by targeting metabolic pathways in TAMS may offer an alternative means of reducing tumor growth and metastasis.

### Macrophage Immunotherapy Approaches Against Cancer

The role of cancer immunotherapy is to stimulate the immune system to recognize, reject, and destroy cancer cells. Cancer immunotherapy with monocytes/macrophages has the goal to polarize macrophages towards a pro-inflammatory response (M1), thus allowing the macrophages and other immune cells to destroy the tumor. Many cytokines and bacterial compounds can achieve this in vitro, although the side effects are typically too severe in vivo. The key is to find a compound with minimal or easily managed patient side effects. Immunotherapy using monocytes/macrophages has been used in past decades and new approaches are being developed every year [64, 65]. Early immunotherapy has established a good foundation for better cancer therapies and increased survival rate in patients treated with immunotherapies [66].

Some approaches to cancer immunotherapy include the use of cytokines or chemokines to recruit activated macrophages and other immune cells to the tumor site which allow for recognition and targeted destruction of the tumor site [67, 68]. IFN-α and IFN-β have been shown to inhibit tumor progression by inducing cell differentiation and apoptosis [69]. Also, IFN treatments are anti-proliferative and can increase S phase time in the cell cycle [70, 71]. Zhang and colleagues performed a study in nude mice using IFN-β gene therapy to target human prostate cancer cells. Their results indicate that adenoviral-delivered IFN-β gene therapy involves macrophages and helps suppress growth and metastasis [72].

The macrophage inhibitory factor (MIF) is another cytokine that can be used in cancer immunotherapy. MIF is usually found in solid tumors and indicates poor prognosis. MIF inhibits aggressive macrophage function and drives macrophages toward an M2 phenotype, which can aid tumor growth and progression. Simpson, Templeton & Cross (2012) found that MIF induces differentiation of myeloid cells, macrophage precursors, into a suppressive population of

myeloid cells that express an M2 phenotype [73]. By targeting MIF, they were able to deplete this suppressive population of macrophages, inhibiting their growth and thus control tumor growth and metastasis [73].

The chemokine receptor type 2, CCR2, is crucial to the recruitment of monocytes to inflammatory sites and it has been shown as a target to prevent the recruitment of macrophages to the tumor site, angiogenesis, and metastasis. Sanford and colleagues (2013) studied a novel CCR2 inhibitor (PF-04136309) in a pancreatic mouse model, demonstrating that the CCR2 inhibitor depleted monocyte/macrophage recruitment to the tumor site, decreased tumor growth and metastasis, and increased antitumor immunity [74]. Another recent study by Schmall et al. showed that macrophages co-cultured with 10 different human lung cancers upregulated CCR2 expression. Moreover, they showed that tumor growth and metastasis were reduced in a lung mouse model treated with a CCR2 antagonist [75].

Other studies have used liposomes to deliver drugs to deplete M2 macrophages from tumors and to stop angiogenesis. Cancer cells that express high levels of IL-1ß grow faster and induce more angiogenesis in vivo. Kimura and colleagues found that macrophages exposed to tumor cells expressing IL-1β produced higher levels of angiogenic factors and chemokines such as vascular endothelial growth factor A (VEG-A), IL-8, monocyte chemoattractant protein 1, etc.,

facilitating tumor growth and angiogenesis [76]. When they used clodronate liposomes to deplete macrophages, they found fewer IL-1B-producing tumor cells. They also found that by inhibiting NF-KB and AP-1 transcription factors in the cancer cells, tumor growth and angiogenesis were reduced. These findings may suggest that macrophages that surround the tumor site may be involved in promoting tumor growth and angiogenesis [76].

Compounds such as methionine enkephalin (MENK) have anti-tumor properties in vivo and in vitro. MENK has the ability to polarize M2 macrophages to M1 macrophages by downregulating CD206 and arginase-1 (M2 markers) while upregulating CD64, MHC-II, and the production of nitric oxide (M1 markers). MENK can also upregulate TNF-α and downregulate IL-10 [77].

Recent studies have focused on bisphosphonates as a potential inhibitor of M2 macrophages. Bisphosphonates are commonly used to treat metastatic breast cancer patients to prevent skeletal complications such as bone resorption [78]. While bisphosphonates stay in the body for short periods of time, bisphosphonates can target osteoclasts, cells in the same family as macrophages, due to their high affinity for hydroxyapatite. Once bisphosphonates bind to the bones, the bone matrix internalizes the bisphosphonates by endocytosis. Once in the cytoplasm, bisphosphonates can inhibit protein prenylation, an event that prevents integrin signaling and

endosomal trafficking, thereby forcing the cell to go apoptotic [69]. Until recently, it was unknown whether bisphosphonates could target tumor-associated macrophages but a recent study by Junankar et al. has shown that macrophages uptake nitrogen-containing bisphosphonate compounds by pinocytosis and phagocytosis, an event that does not occur in epithelial cells surrounding the tumor [79]. Forcing TAMs to go apoptotic using bisphosphonates could reduce angiogenesis and metastasis.

Additional approaches to cancer immunotherapy include the use of biomaterials that may elicit an immune response. Cationic polymers are used in immunotherapy because of their reactivity once dissolved in water. Chen et al. used cationic polymers including PEI, polylysine, cationic dextran and cationic gelatin to produce a strong Th1 immune response [77]. They were also able to induce proliferation of CD4+cells and secretion of IL-12 typical of M1 macrophages [77]. Huang and colleagues also used biomaterials to trigger TAMs to produce an anti-tumor response by targeting TLR4 [80]. This study found that TAMs were able to polarize to an M1 phenotype and express IL-12. They found that these cationic molecules have direct tumoricidal activity and demonstrate tumor reduction in mice [80].

### TLR4

Toll-like receptor 4 is a protein in humans that is encoded by the TLR4 gene. TLR 4 detects lipopolysaccharide (LPS) on gram negative bacteria and thus plays a fundamental role in the recognition of danger and the activation of the innate immune system (FIG. 7). It cooperates with LY96 (MD-2) and CD14 to mediate signal transduction when macrophages are induced by LPS. The cytoplasmic domain of TLR4 is responsible for the activation of M1 macrophages when they detect the presence of LPS. This is the functional portion of the receptor that would be coupled to the MOTO-CAR (i.e., chimeric receptor) to induce activation of the monocyte/macrophage when the CAR binds its target protein.

The adaptor proteins MyD88 and TIRAP contribute to the activation of several and possibly all pathways via direct interactions with TLR4's Toll/interleukin-1 receptor (IL-1R) (TIR) domain. However, additional adaptors that are required for the activation of specific subsets of pathways may exist, which could contribute to the differential regulation of target genes.

### Thymidine Kinase

Human Thymidine Kinase 1 (TK1) is a well-known nucleotide salvage pathway enzyme that has largely been studied in the context of its overexpression in tumors. Since TK1 was initially popularized by its expression in the serum of cancer patients (sTK), its diagnostic and prognostic potential has been studied extensively. For example, several studies have demonstrated that sTK1 in many different cancer patients is elevated in a stage-like manner with a higher level of TK1 indicating a more advanced tumor [81].

Other studies have investigated the prognostic potential of TK1. One such study demonstrates that the TK1 levels in primary breast tumors can be used to predict recurrence. Other exciting TK1 prognostic studies show significant reductions in sTK1 levels when patients respond to treatment while sTK1 levels continue to rise in patients who do not appear to respond to their treatment. It is also known that sTK1 levels begin to rise prior to recurrence and noted in some cases sTK1 levels could predict recurrence “1-6 months before the onset of clinical symptoms.” Several other studies confirm the rich potential of TK1 as a diagnostic and prognostic indicator of cancer [82].

Although the diagnostic and prognostic potential of TK1 has been well established, the therapeutic potential of TK1 remains veiled in comparison. While it is true that HSV-TK has been used in gene therapy and PET imaging utilizes TK1 to identify proliferating cancer cells, few, if any studies address the possibility of a TK1 immunotherapy. Perhaps this is primarily because TK1 is a known cytosolic protein. It has been recently discovered that TK1 is expressed not only in cancer cells but also on the surface membrane of multiple tumor types and is therefore a very viable target for tumor immunotherapy.

### BRIEF SUMMARY

Described herein are chimeric receptors. Chimeric receptors comprise a cytoplasmic domain; a transmembrane domain; and an extracellular domain. In embodiments, the cytoplasmic domain comprises a cytoplasmic portion of a receptor that when activated polarizes a macrophage. In further embodiments, a wild-type protein comprising the cytoplasmic portion does not comprise the extracellular domain of the chimeric receptor (see,.e.g., FIG. 21). In embodiments, the binding of a ligand to the extracellular domain of the chimeric receptor activates the intracellular portion of the chimeric receptor (see,.e.g., FIG. 22). Activation of the intracellular portion of the chimeric receptor may polarize the macrophage into an M1 or M2 macrophage (see,.e.g., FIGS. 23 and 24 (A) and 25).

In certain embodiments, the extracellular domain may comprise an antibody or a fragment there of that specifically binds to a ligand. In embodiments, the chimeric receptor may contain a linker. In embodiments, the chimeric receptor may contain a hinge region.

Further embodiments include cells comprising a chimeric receptor or nucleic acids encoding a chimeric receptor.

Embodiments include methods of polarizing a macrophage by contacting a macrophage comprising a chimeric receptor with a ligand for the extracellular domain of the chimeric receptor; binding the ligand to the extracellular domain of the chimeric receptor. The binding of the ligand to the extracellular domain of the chimeric receptor activates the cytoplasmic portion and the activation of the cytoplasmic portion polarizes the macrophage.

These and other aspects of the disclosure will become apparent to the skilled artisan in view of the teachings contained herein.

### BRIEF DESCRIPTION OF THE SEVERAL VIEWS OF THE DRAWINGS

FIG. 1A depicts a block diagram of the order of elements in the chimeric receptor TK1-MOTO1. FIG. 1B depicts the sequence of TK1-MOTO1 (SEQ ID NO:35). Amino acids 1-18 are a signal peptide (SP), amino acids 19-275 are an anti-TK1 ScFv, amino acids 276-290 are a GS linker, amino acids 291-313 are a TLR4 transmembrane domain, and amino acids 314-496 are a TLR4 cytosolic domain.

FIG. 2A depicts a block diagram of the order of elements in the chimeric receptor TK1-MOTO2. FIG. 2B depicts the sequence of TK1-MOTO2 (SEQ ID NO:36). Amino acids 1-18 are a signal peptide (SP), amino acids 19-275 are an anti-TK1 ScFv, amino acids 276-290 are a GS linker, amino acids 291-295 are a LRR short hinge, amino acids 296-318 are a TLR4 transmembrane domain, and amino acids 319-500 are a TLR4 cytosolic domain.

FIG. 3A depicts a block diagram of the order of elements in the chimeric receptor TK1-MOTO3. FIG. 3B depicts the sequence of TK1-MOTO3 (SEQ ID NO:37). Amino acids 1-18 are a signal peptide (SP), amino acids 19-275 are an anti-TK1 ScFv, amino acids 276-290 are a GS linker, amino acids 291-345 are a LRR long hinge, amino acids 346-368 are a TLR4 transmembrane domain, and amino acids 269-501 are a TLR4 cytosolic domain.

FIG. 4A depicts a block diagram of the order of elements in the chimeric receptor TK1-MOTO4. FIG. 4B depicts the sequence of TK1-MOTO4 (SEQ ID NO:38). Amino acids 1-18 are a signal peptide (SP), amino acids 19-275 are an anti-TK1 ScFv, amino acids 276-290 are a GS linker, amino acids 291-302 are an IgG4 short hinge, amino acids 303-325 are a TLR4 transmembrane domain, and amino acids 326-508 are a TLR4 cytosolic domain.

FIG. 5A depicts a block diagram of the order of elements in the chimeric receptor TK1-MOTO5. FIG. 5B depicts the sequence of TK1-MOTO5 (SEQ ID NO:39). Amino acids 1-18 are a signal peptide (SP), amino acids 19-275 are an anti-TK1 ScFv, amino acids 276-290 are a GS linker, amino acids 291-409 are an IgG 119 amino acid medium hinge, amino acids 410-432 are a TLR4 transmembrane domain, and amino acids 433-615 are a TLR4 cytosolic domain.

FIG. 6A depicts a block diagram of the order of elements in the chimeric receptor TK1-MOTO6. FIG. 6B depicts the sequence of TK1-MOTO6 (SEQ ID NO:40). Amino acids 1-18 are a signal peptide (SP), amino acids 19-275 are an anti-TK1 ScFv, amino acids 276-290 are a GS linker, amino acids 291-518 are an IgG4 long hinge, amino acids 519-541 are a TLR4 transmembrane domain, and amino acids 542-724 are a TLR4 cytosolic domain.

FIG. 7A depicts a block diagram of the order of elements in the chimeric receptor TK1-MOTO7. FIG. 7B depicts the sequence of TK1-MOTO7 (SEQ ID NO:41). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-381 are a TLR4 transmembrane domain, and amino acids 382-564 are a TLR4 cytosolic domain.

FIG. 8A depicts a block diagram of the order of elements in the chimeric receptor TK1-MOTO8. FIG. 8B depicts the sequence of TK1-MOTO8 (SEQ ID NO:42). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-381 are a TLR4 transmembrane domain, and amino acids 382-564 are a TLR4 cytosolic domain.

FIG. 9A depicts a block diagram of the order of elements in the chimeric receptor TK1-MO-FCGRA-CAR-1. FIG. 9B depicts the sequence of TK1-MO-FCGRA-CAR-1 (SEQ ID NO: 43). Amino acids 1-18 are a signal peptide (SP), amino acids 19-275 are an anti-TK1 ScFv, amino acids 276-290 are a GS linker, amino acids 291-311 are a FCGR3A transmembrane domain, amino acids 312-336 are a FCGR3A cytosolic domain, and amino acids 337-378 are a FCER1G cytosolic domain.

FIG. 10A depicts a block diagram of the order of elements in the chimeric receptor TK1-MO-FCGRA-CAR-2. FIG. 10B depicts the sequence of TK1-MO-FCGRA-CAR-2 (SEQ ID NO: 44). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-379 are a FCGR3A transmembrane domain, amino acids 380-404 are a FCGR3A cytosolic domain, and amino acids 405-446 are a FCER1G cytosolic domain.

FIG. 11A depicts a block diagram of the order of elements in the chimeric receptor TK1-MO-FCGRA-CAR-3. FIG. 11B depicts the sequence of TK1-MO-FCGRA-CAR-3 (SEQ ID NO: 45). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-379 are a FCGR3A transmembrane domain, amino acids 380-404 are a FCGR3A cytosolic domain, and amino acids 405-446 are a FCER1G cytosolic domain.

FIG. 12A depicts a block diagram of the order of elements in the chimeric receptor TK1-MO-FCGRA-CAR-4. FIG. 12B depicts the sequence of TK1-MO-FCGRA-CAR-4 (SEQ ID NO: 46). Amino acids 1-18 are a signal peptide (SP), amino acids 19-275 are an anti-TK1 ScFv, amino acids 276-290 are a GS linker, amino acids 291-303 are a IgG4 short hinge, amino acids 304-324 are a FCGR3A transmembrane domain, amino acids 325-349 are a FCGR3A cytosolic domain, and amino acids 350-391 are a FCER1G cytosolic domain.

FIG. 13A depicts a block diagram of the order of elements in the chimeric receptor TK1-MO-FCGRA-CAR-5. FIG. 13B depicts the sequence of TK1-MO-FCGRA-CAR-5 (SEQ ID NO: 47). Amino acids 1-18 are a signal peptide (SP), amino acids 19-275 are an anti-TK1 ScFv, amino acids 276-290 are a GS linker, amino acids 291-409 are a IgG4 119 amino acid hinge, amino acids 410-430 are a FCGR3A transmembrane domain, amino acids 431-455 are a FCGR3A cytosolic domain, and amino acids 456-497 are a FCER1G cytosolic domain.

FIG. 14A depicts a block diagram of the order of elements in the chimeric receptor TK1-MO-FCGRA-CAR-6. FIG. 14B depicts the sequence of TK1-MO-FCGRA-CAR-6 (SEQ ID NO: 48). Amino acids 1-18 are a signal peptide (SP), amino acids 19-275 are an anti-TK1 ScFv, amino acids 276-290 are a GS linker, amino acids 291-519 are a IgG4 long hinge, amino acids 520-540 are a FCGR3A transmembrane domain, amino acids 541-565 are a FCGR3A cytosolic domain, and amino acids 566-607 are a FCER1G cytosolic domain.

FIG. 15A depicts a block diagram of the order of elements in the chimeric receptor TK1-MO-FCG2A-CAR-1. FIG. 15B depicts the sequence of TK1-MO-FCG2A-CAR-1 (SEQ ID NO: 49). Amino acids 1-18 are a signal peptide (SP), amino acids 19-275 are an anti-TK1 ScFv, amino acids 276-290 are a GS linker, amino acids 291-312 are a FCGR2A transmembrane domain, amino acids 313-390 are a FCGR2A cytosolic domain.

FIG. 16A depicts a block diagram of the order of elements in the chimeric receptor TK1-MO-FCG2A-CAR-2. FIG. 16B depicts the sequence of TK1-MO-FCG2A-CAR-2 (SEQ ID NO: 50). Amino acids 1-18 are a signal peptide (SP), amino acids 19-275 are an anti-TK1 ScFv, amino acids 276-290 are a GS linker, amino acids 291-358 are mutated CD8 hinge with C339S and C356S, amino acids 359-380 are a FCGR2A transmembrane domain, amino acids 381-458 are a FCGR2A cytosolic domain.

FIG. 17A depicts a block diagram of the order of elements in the chimeric receptor TK1-MO-FCG2A-CAR-3. FIG. 17B depicts the sequence of TK1-MO-FCG2A-CAR-3 (SEQ ID NO: 51). Amino acids 1-18 are a signal peptide (SP), amino acids 19-275 are an anti-TK1 ScFv, amino acids 276-290 are a GS linker, amino acids 291-358 are a portion of a CD8 hinge, amino acids 359-380 are a FCGR2A transmembrane domain, amino acids 381-458 are a FCGR2A cytosolic domain.

FIG. 18A depicts a block diagram of the order of elements in the chimeric receptor TK1-MO-FCG2A-CAR-4. FIG. 18B depicts the sequence of TK1-MO-FCG2A-CAR-4 (SEQ ID NO: 52). Amino acids 1-18 are a signal peptide (SP), amino acids 19-275 are an anti-TK1 ScFv, amino acids 276-290 are a GS linker, amino acids 291-303 are a IgG4 short hinge, amino acids 304-325 are a FCGR2A transmembrane domain, amino acids 326-403 are a FCGR2A cytosolic domain.

FIG. 19A depicts a block diagram of the order of elements in the chimeric receptor TK1-MO-FCG2A-CAR-5. FIG. 19B depicts the sequence of TK1-MO-FCG2A-CAR-5 (SEQ ID NO: 53). Amino acids 1-18 are a signal peptide (SP), amino acids 19-275 are an anti-TK1 ScFv, amino acids 276-290 are a GS linker, amino acids 291-409 are a IgG4 119 amino acid hinge, amino acids 410-431 are a FCGR2A transmembrane domain, amino acids 432-509 are a FCGR2A cytosolic domain.

FIG. 20A depicts a block diagram of the order of elements in the chimeric receptor TK1-MO-FCG2A-CAR-6. FIG. 20B depicts the sequence of TK1-MO-FCG2A-CAR-6 (SEQ ID NO: 54). Amino acids 1-18 are a signal peptide (SP), amino acids 19-275 are an anti-TK1 ScFv, amino acids 276-290 are a GS linker, amino acids 291-519 are a IgG4 long hinge, amino acids 520-541 are a FCGR2A transmembrane domain, amino acids 542-619 are a FCGR2A cytosolic domain.

FIG. 21 is a schematic illustrating a chimeric receptor.

FIG. 22 is a schematic showing a macrophage expressing a chimeric receptor. As depicted, the chimeric receptor comprises the cytosolic domain of a toll like receptors, a transmembrane domain, and a ScFv specific for a ligand. The arrows depict signaling to polarize the macrophage upon the ScFv binding the ligand.

FIG. 23 is a schematic showing different macrophage receptors that could be utilized to build a chimeric receptor.

FIGS. 24A through 24C. FIG. 24A is a schematic showing the Fc Gamma Receptor III signaling cascade leading to cell activation. FIG. 24B is a schematic showing the Fc Gamma Receptor III signaling cascade leading to inhibition of calcium flux and proliferation. FIG. 24C is a schematic showing the Fc Gamma Receptor III signaling cascade leading to apoptosis.

FIG. 25 is a schematic illustrating the Toll Like Receptor Signaling cascade.

FIG. 26 presents graphs illustrating flow cytometry confirming that an expressed antibody fragment binds the ligand of interest.

FIG. 27 presents two images showing a phenotype change in macrophages after transduction with a chimeric receptor.

FIG. 28 presents two images confirming the expression of a chimeric receptor in monocytes.

FIG. 29 presents three scatter plots of fluorescence activated cell sorting demonstrating the expression of dTomato. The left most plot shows a control wherein only 0.58% of cells show fluorescence which would indicate expression of dTomato. The right two plots show a transduction efficiency of 27.1 percent after transduction.

FIG. 30 presents six scatter plots of fluorescence activated cell sorting demonstrating the retention of dye (Alexa 647), and the expression of CD80, CD163, CD206, and CD14 in macrophages transduced with a chimeric receptor.

FIG. 31 presents a histogram demonstrating the relative expression levels of CD80, CD163, CD206, and CD14 in macrophages transduced with a chimeric receptor.

FIG. 32 presents six images of transduced macrophages expressing a chimeric receptor detecting, attacking, and inducing cell death in a lung cancer cell line (NCI-H460).

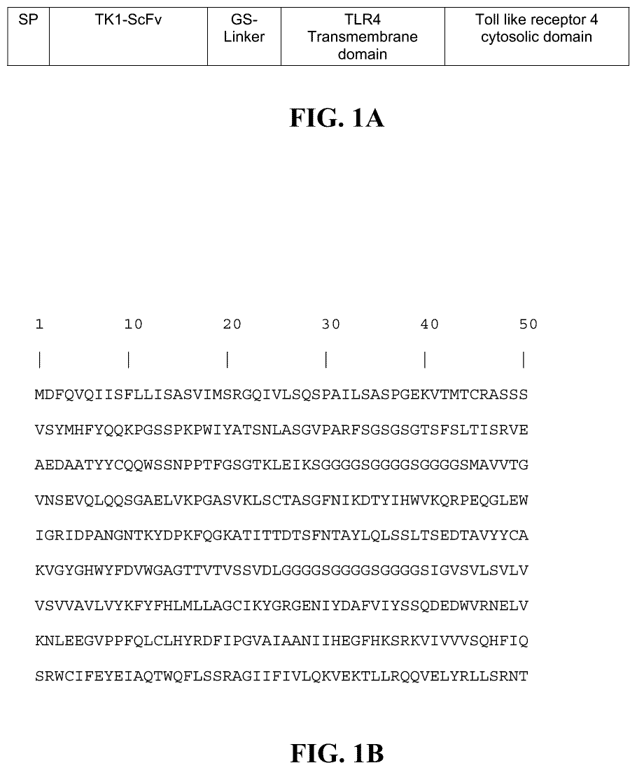

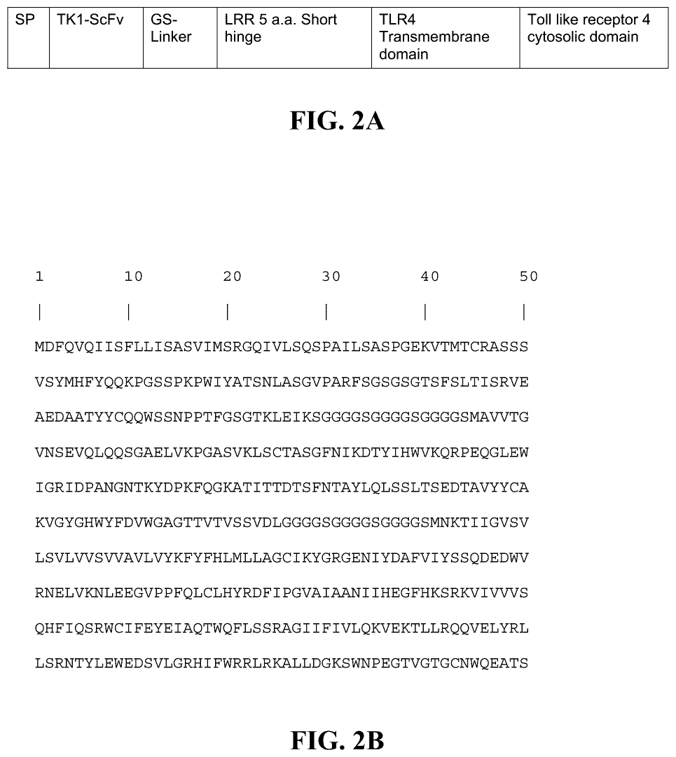

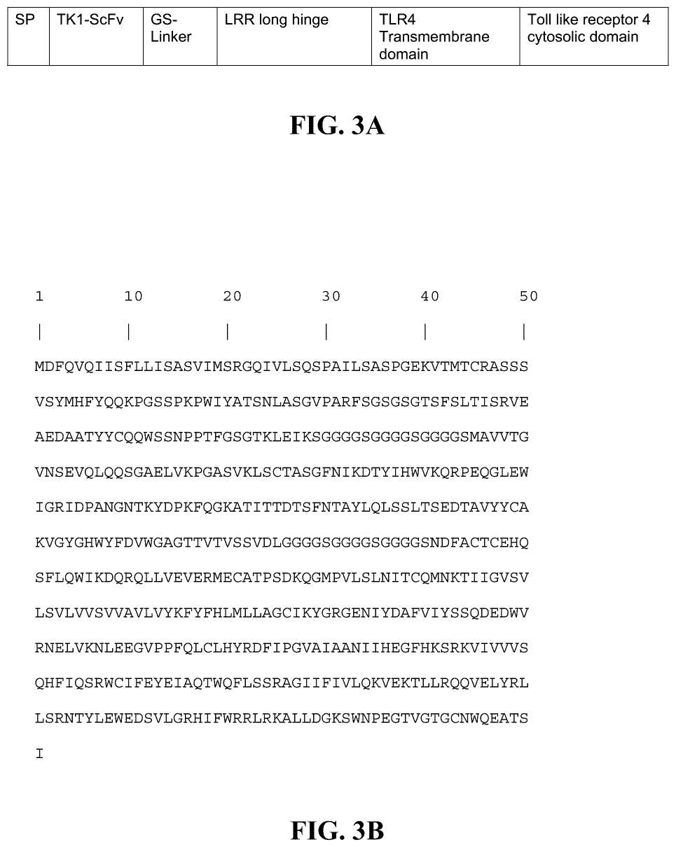

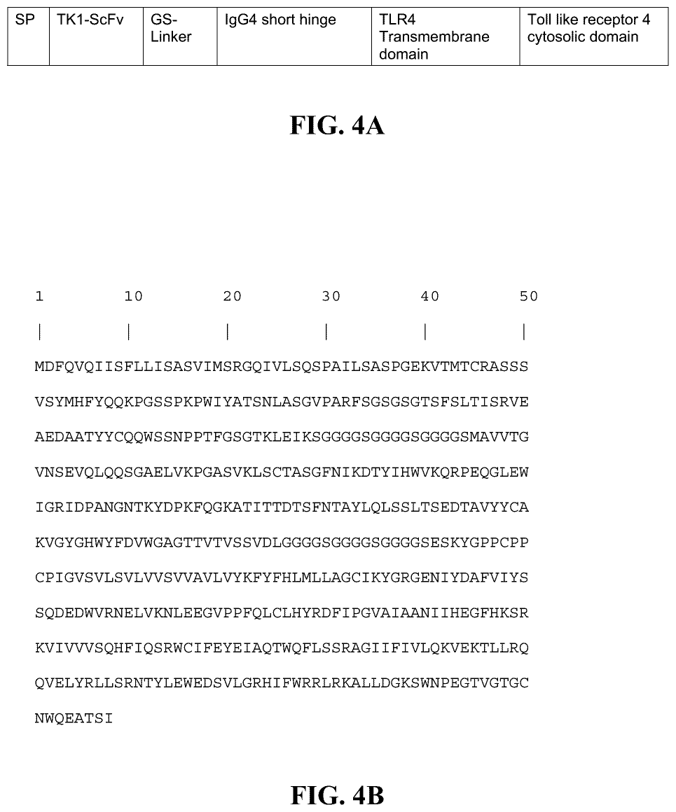

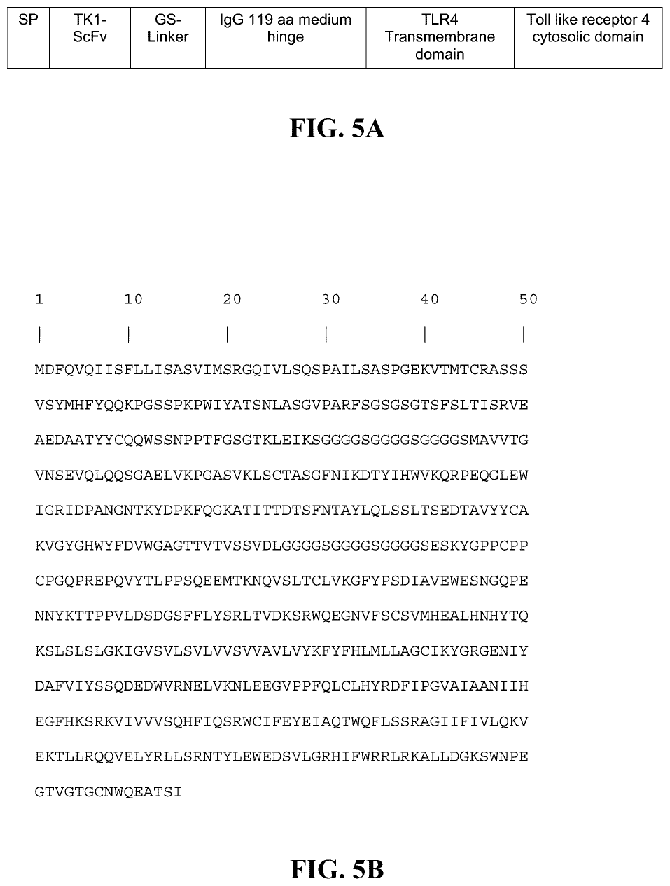

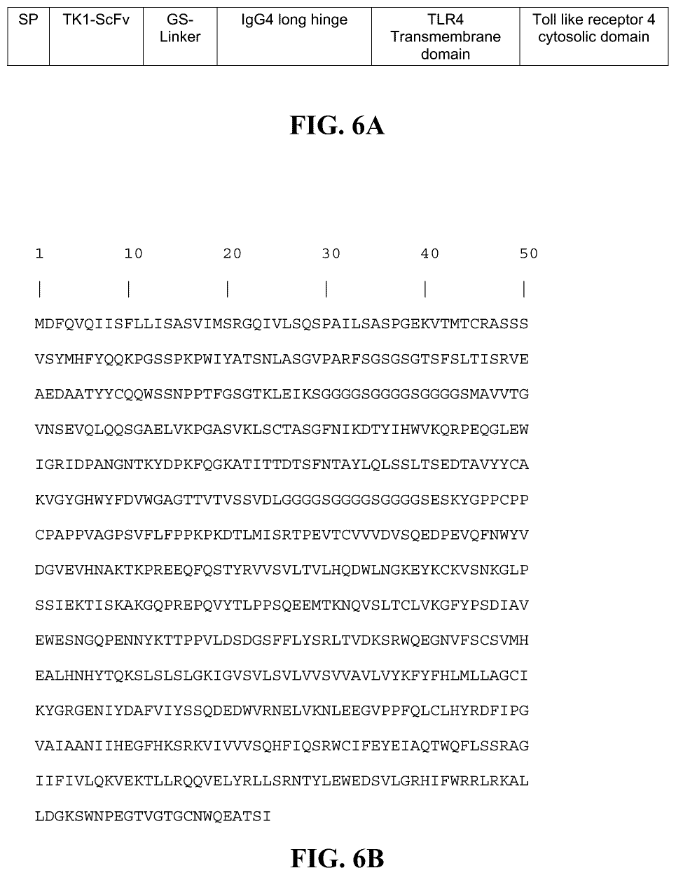

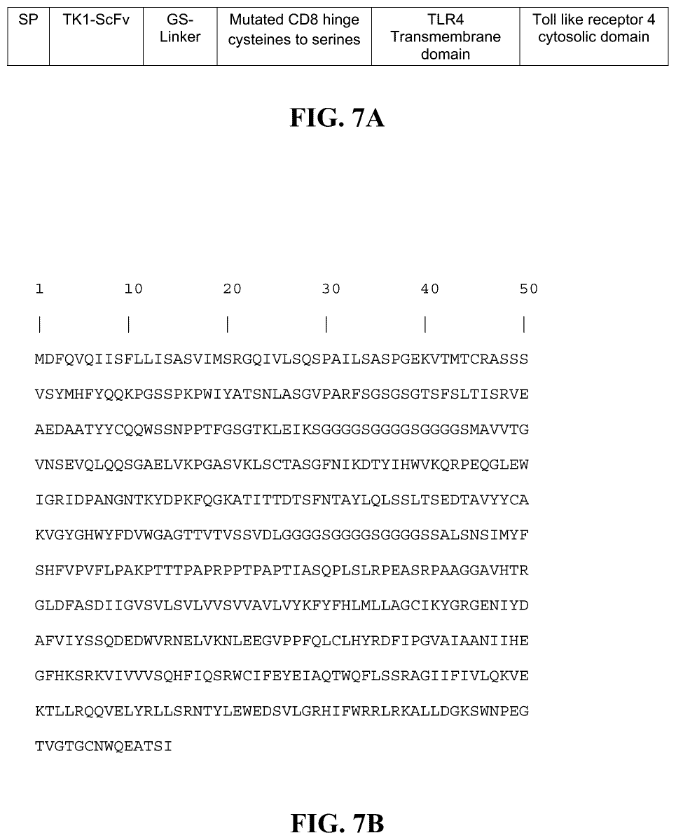

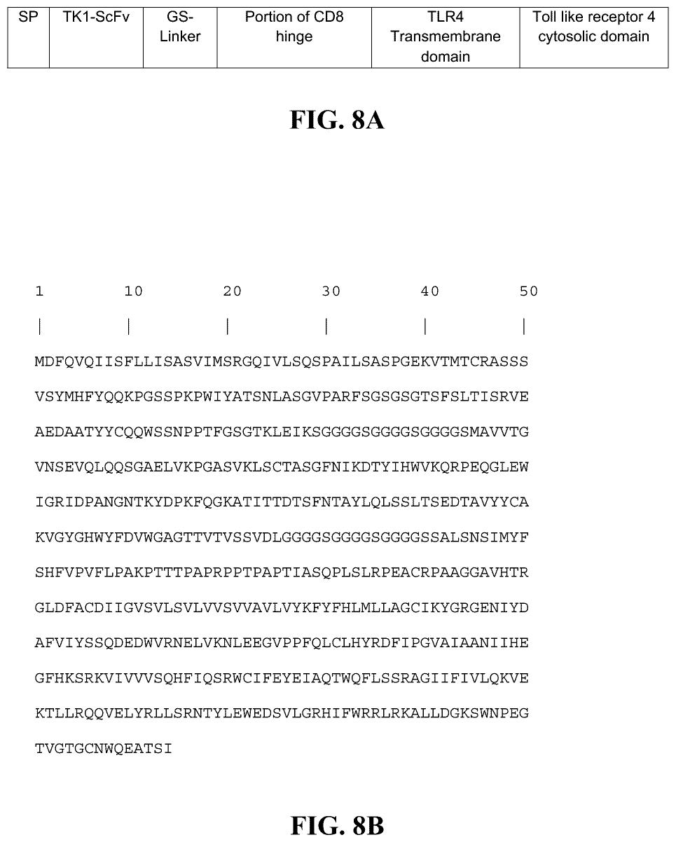

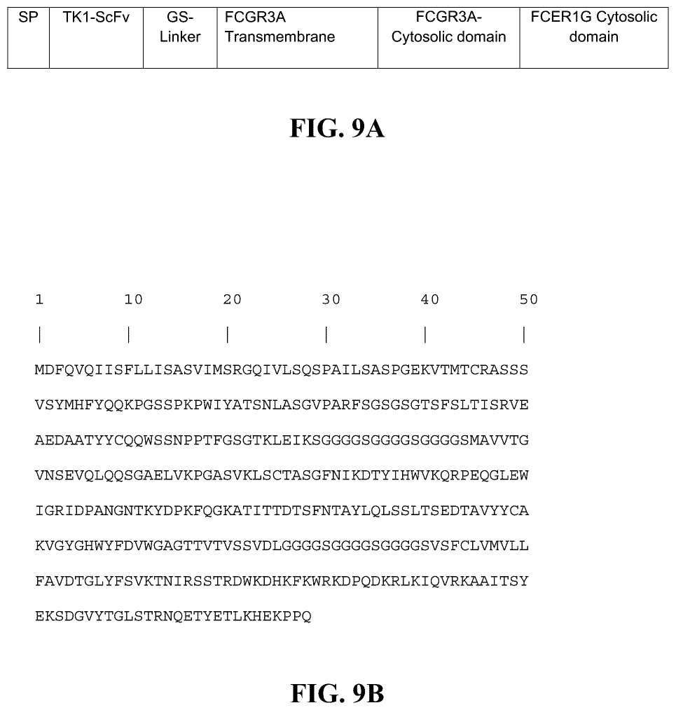

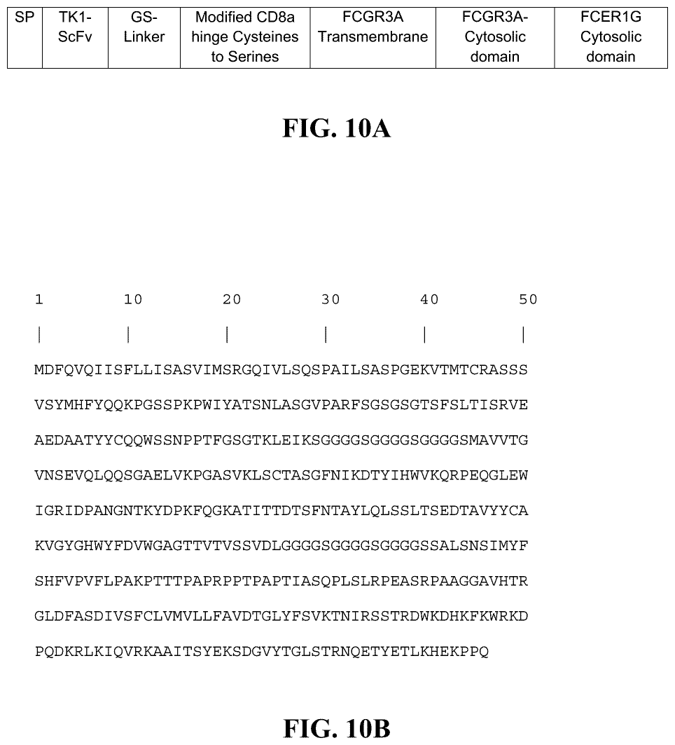

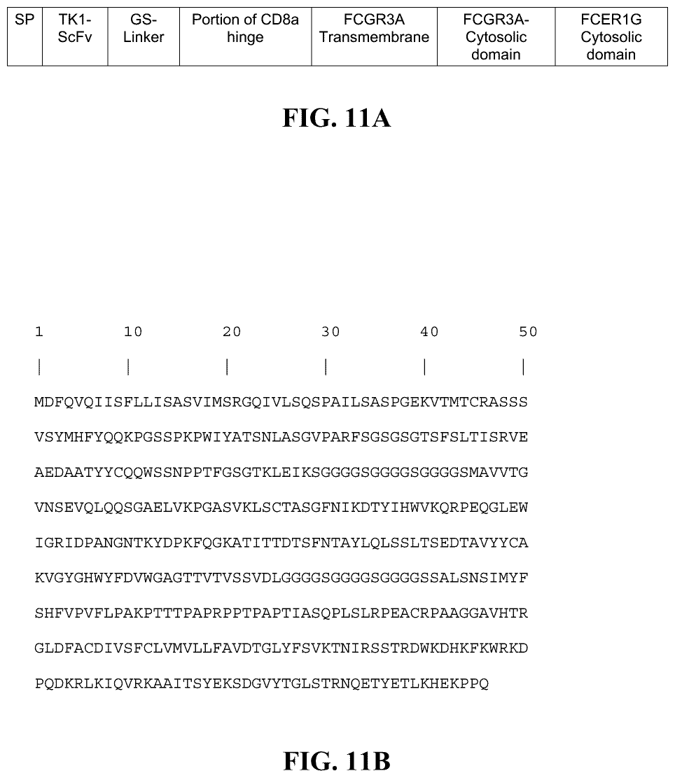

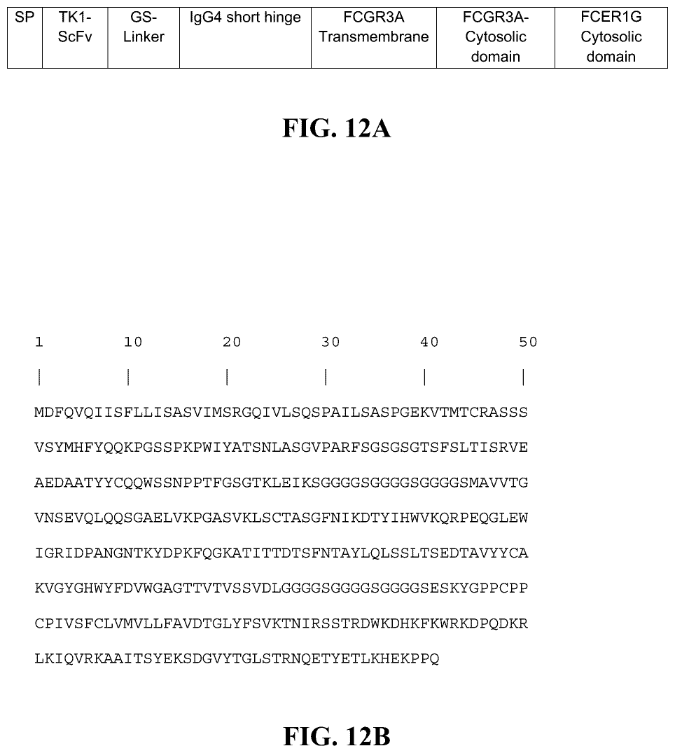

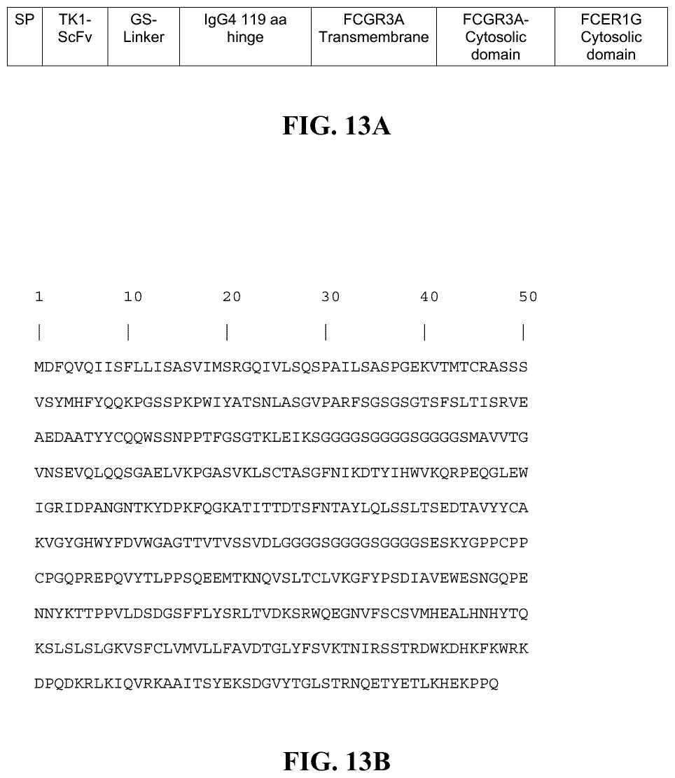

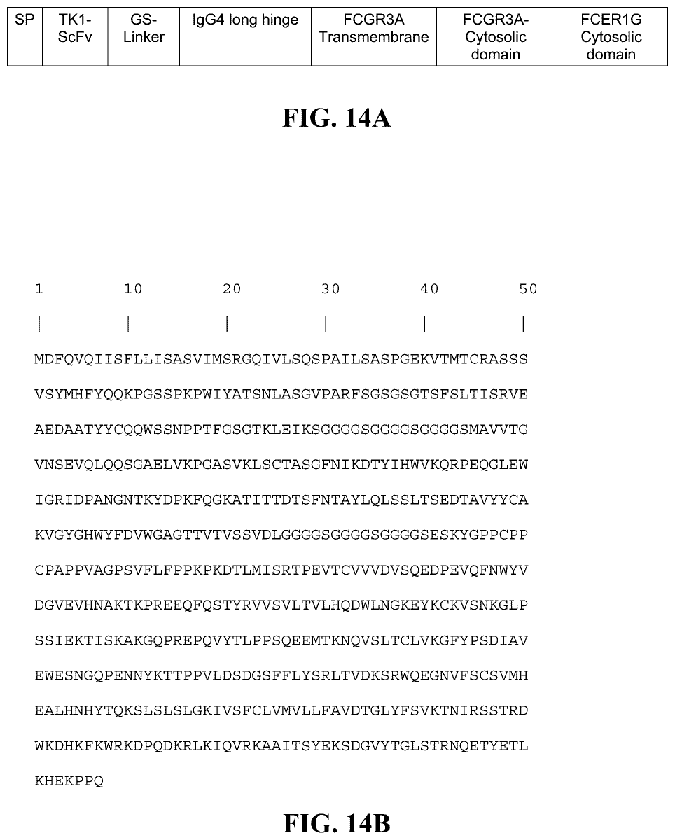

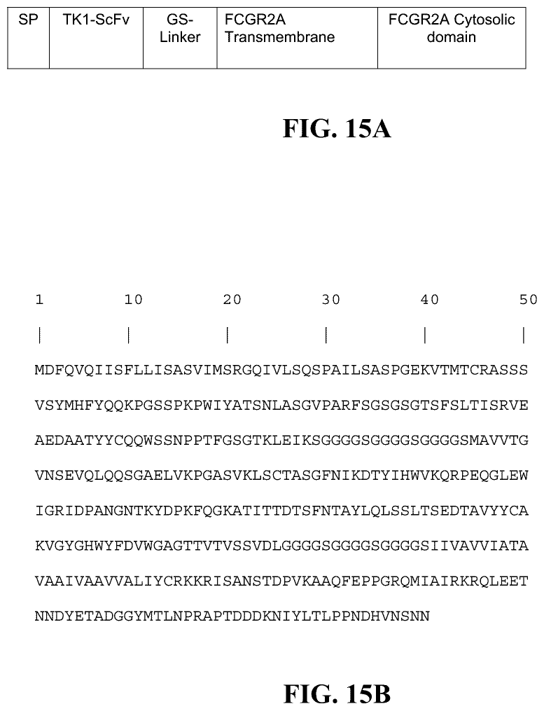

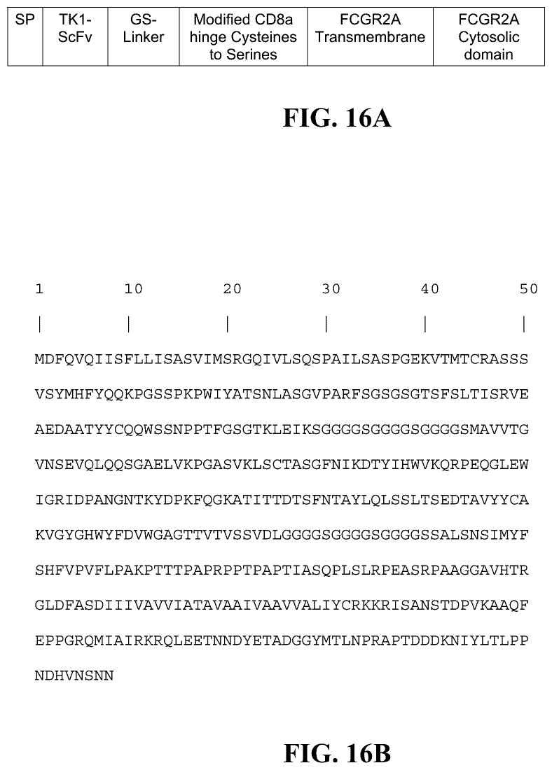

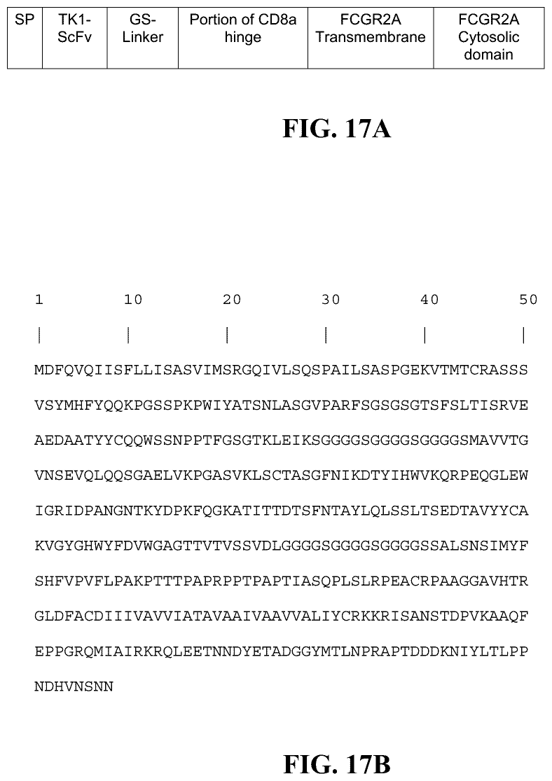

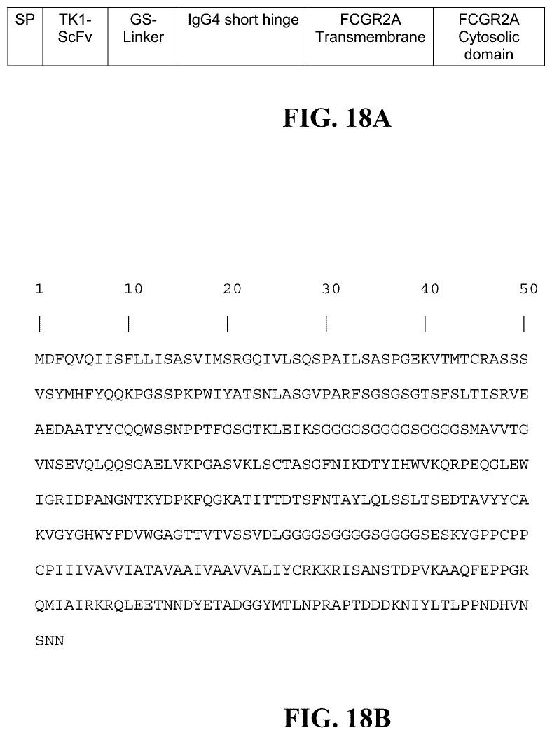

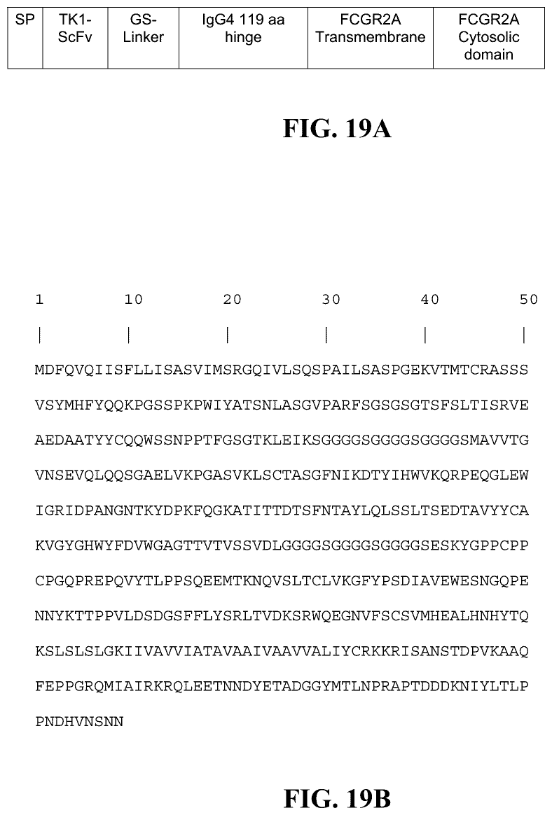

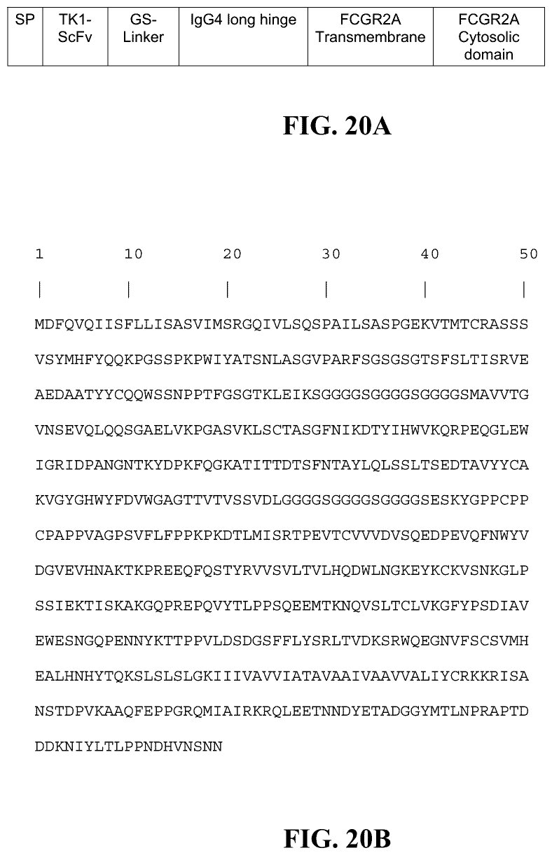

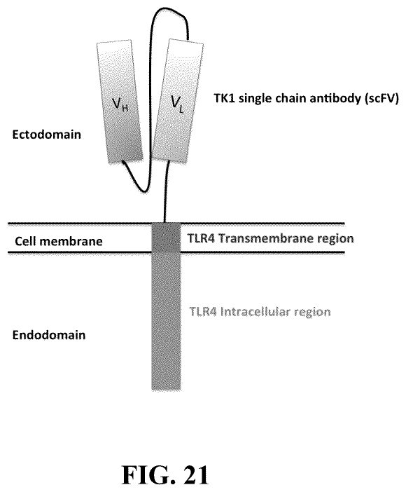

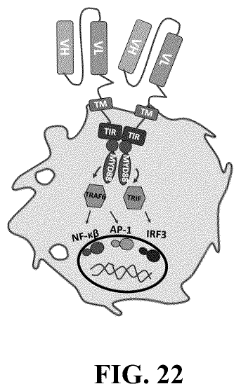

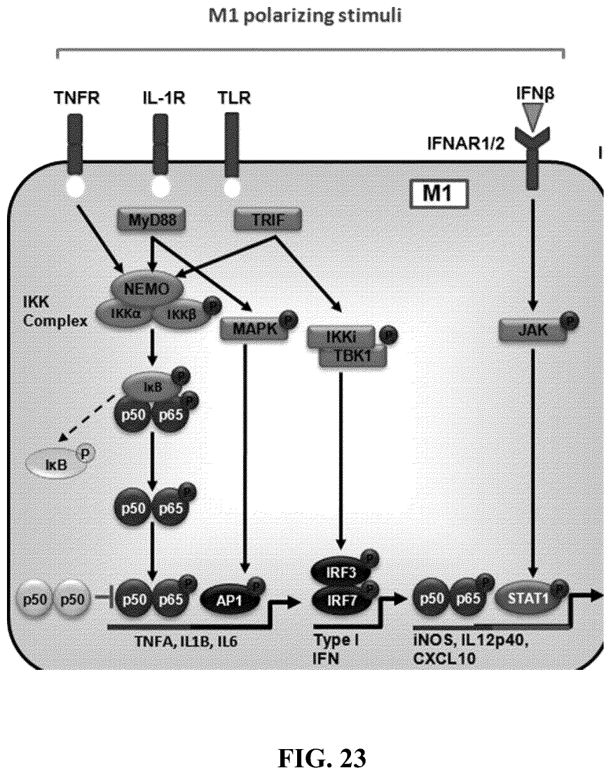

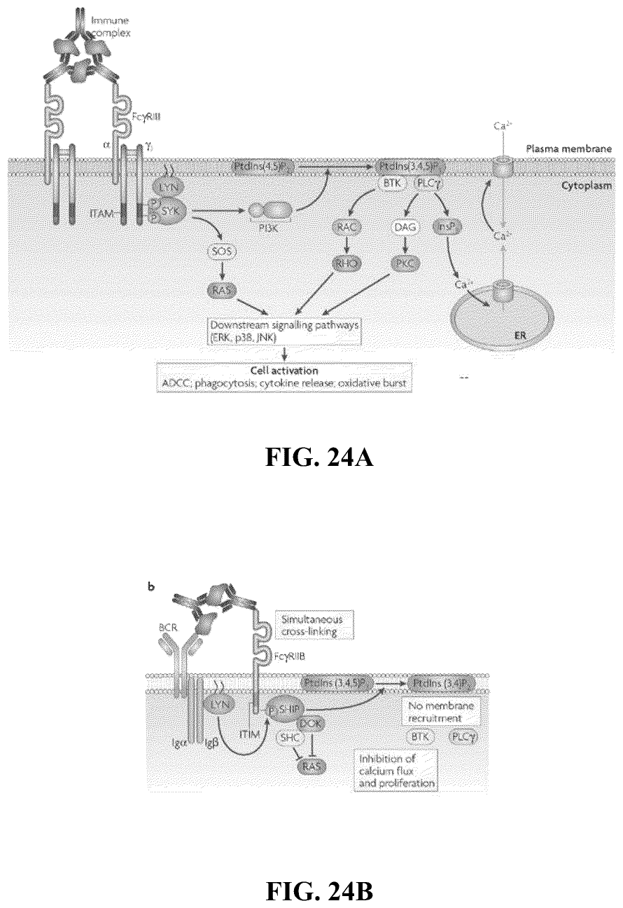

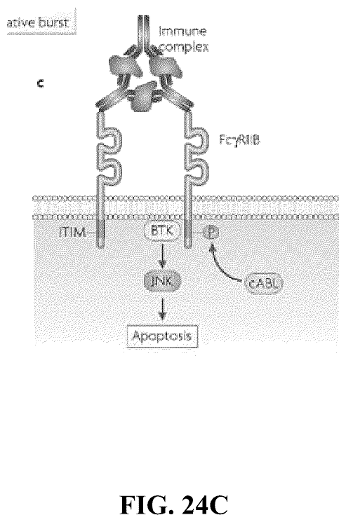

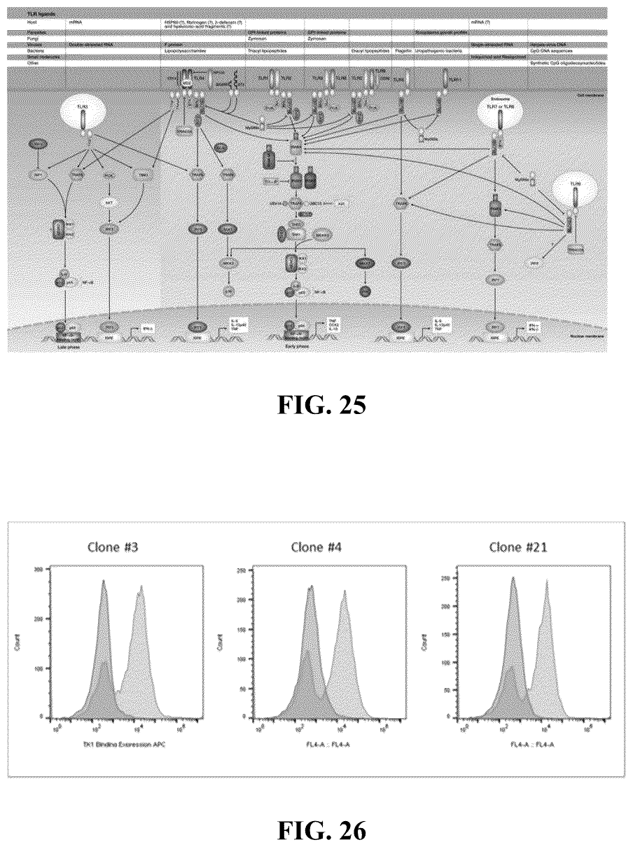

### DETAILED DESCRIPTION

Described herein are chimeric receptors. Chimeric receptors comprise a cytoplasmic domain; a transmembrane domain; and an extracellular domain. In embodiments, the cytoplasmic domain comprises a cytoplasmic portion of a receptor that when activated polarizes a macrophage. In further embodiments, a wild-type protein comprising the cytoplasmic portion does not comprise the extracellular domain of the chimeric receptor. In embodiments, the binding of a ligand to the extracellular domain of the chimeric receptor activates the intracellular portion of the chimeric receptor. Activation of the intracellular portion of the chimeric receptor may polarize the macrophage into an M1 or M2 macrophage.

In certain embodiments, the cytoplasmic portion of the chimeric receptor may comprise a cytoplasmic domain from a toll-like receptor, myeloid differentiation primary response protein (MYD88) (SEQ ID NO: 19), toll-like receptor 3 (TLR3) (SEQ ID NO:1), toll-like receptor 4 (TLR4) (SEQ ID NO:3), toll-like receptor 7 (TLR7) (SEQ ID NO:7), toll-like receptor 8 (TLR8) (SEQ ID NO:9), toll-like receptor 9 (TLR9) (SEQ ID NO:11), myelin and lymphocyte protein (MAL) (SEQ ID NO:21), interleukin-1 receptor-associated kinase 1 (IRAK1) (SEQ ID NO:23), low affinity immunoglobulin gamma Fc region receptor III-A (FCGR3A) (SEQ ID NO:15), low affinity immunoglobulin gamma Fc region receptor II-a (FCGR2A) (SEQ ID NO:13), high affinity immunoglobulin epsilon receptor subunit gamma (FCER1G) (SEQ ID NO:19), or sequences having at least 90% sequence identity to a cytoplasmic domain of any one of the foregoing. In certain embodiments, the cytoplasmic portion is not a cytoplasmic domain from a toll-like receptor, FCGR3A, IL-1 receptor, or IFN-gamma receptor. In embodiments, the cytosolic portion can be any polypeptide that, when activated, will result in the polarization of a macrophage.

In further embodiments, examples of ligands which bind to the extracellular domain may be, but are not limited to, Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoictin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171. In certain embodiments, the ligand is not TK1 or HPRT.

Antibodies which may be adapted to generate extracellular domains of a chimeric receptor are well known in the art and are commercially available. Examples of commercially available antibodies include, but are not limited to: anti-HGPRT, clone 13H11.1 (EMD Millipore), anti-ROR1 (ab135669) (Abcam), anti-MUCI [EP1024Y] (ab45167) (Abcam), anti-MUC16 [X75] (ab1107) (Abcam), anti-EGFRvIII [L8A4] (Absolute antibody), anti-Mesothelin [EPR2685 (2)] (ab134109) (Abcam), HER2 [3B5] (ab16901) (Abcam), anti-CEA (LS-C84299-1000) (LifeSpan BioSciences), anti-BCMA (ab5972) (Abcam), anti-Glypican 3 [9C2] (ab 129381) (Abcam), anti-FAP (ab53066) (Abcam), anti-EphA2 [RM-0051-8F21] (ab73254) (Abcam), anti-GD2 (LS-C546315) (LifeSpan BioSciences), anti-CD19 [2E2B6B10] (ab31947) (Abcam), anti-CD20 [EP459Y] (ab78237) (Abcam), anti-CD30 [EPR4102] (ab134080) (Abcam), anti-CD33 [SP266] (ab199432) (Abcam), anti-CD123 (ab53698) (Abcam), anti-CD133 (BioLegend), anti-CD123 (1A3H4) ab181789 (Abcam), and anti-CD171 (L1.1) (Invitrogen antibodies). Techniques for creating antibody fragments, such as ScFvs, from known antibodies are routine in the art. Further, generating sequences encoding such fragments and recombinantly including them in as part of a polynucleotide encoding a chimeric protein is also routine in the art.

In certain embodiments, the extracellular domain may comprise an antibody or a fragment there of that specifically binds to a ligand. Examples of antibodies and fragments thereof include, but are not limited to IgAs, IgDs, IgEs, IgGs, IgMs, Fab fragments, F (ab′)₂ fragments, monovalent antibodies, ScFv fragments, scRv-Fc fragments, IgNARs, hclgGs, VhH antibodies, nanobodies, and alphabodies. In additional embodiments, the extracellular domain may comprise any amino acid sequence that allows for the specific binding of a ligand, including, but not limited to, dimerization domains, receptors, binding pockets, etc.

In embodiments, the chimeric receptor may contain a linker. Without limitation, the linker may be located between the extracellular domain and the transmembrane domain of the chimeric receptor. Without limitation, the linker may be a G linker, a GS linker, a G4S linker, an EAAAK linker, a PAPAP linker, or an (Ala-Pro), linker. Other examples of linkers are well known in the art.

In embodiments, the chimeric receptor may contain a hinge region. Without limitation, the hinge region may be located between the extracellular domain and the transmembrane domain of the chimeric receptor. In further embodiments, the hinge region may be located between a linker and the transmembrane domain. Without limitation, the linker may be a leucine rich repeat (LRR), or a hinge region from a toll-like receptor, an IgG, IgG4, CD8m or FcγIIIa-hing. In embodiments, cysteines in the hinge region may be replaced with serines. Other examples of hinge regions are well known in the art.

Chimeric receptors as described herein may comprise one or more of SEQ ID NOS: 1, 3, 4, 5, 7, 9, 11, 13, 15, 17, 19, 21, 23, and 25-34, fragments of any of thereof, and/or polypeptides having at least 90% sequence identity to at least one of SEQ ID NOS: 1, 3, 4, 5, 7, 9, 11, 13, 15, 17, 19, 21, 23, and 25-34 or fragments thereof. Examples of chimeric receptors include, but are not limited to, SEQ ID NOS: 35-54, or a homologue or fragment thereof. In another embodiment, the polypeptide comprises an amino acid sequence selected from the group consisting of a polypeptide having at least 90% sequence identity to at least one of SEQ ID NOS: 35-54.

Embodiments include nucleic acid sequences comprising a nucleic acid sequence encoding a chimeric receptor as described above. Examples of such nucleic acids may comprise one or more of SEQ ID NOS: 2, 6, 8, 10, 12, 14, 16, 18, 20, 22, and 24, fragments of any of thereof, and/or nucleic acids having at least 90% sequence identity to at least one of SEQ ID NOS: 2, 6, 8, 10, 12, 14, 16, 18, 20, 22, and 24 or fragments thereof. Further examples include nucleic acids encoding one or more of SEQ ID NOS: 24-54 and fragments of any of thereof.

In embodiments, the chimeric receptors may be glycosylated, pegylated, and/or otherwise post-translationally modified. Further, the nucleic acid sequence may be part of a vector. By way of example, the vector may be a plasmid, phage, cosmid, artificial chromosome, viral vector, AAV vector, adenoviral vector, or lentiviral vector. In certain embodiments, a nucleic acid encoding a chimeric receptor may be operably linked to a promoter and/or other regulatory sequences (e.g., enhancers, silencers, insulators, locus control regions, cis-acting elements, etc.).

Further embodiments include cells comprising a chimeric receptor or nucleic acids encoding a chimeric receptor. Non-limiting examples of such cells include myeloid cells, myeloid progenitor cells, monocytes, neutrophils, basophils, cosinophils, megakaryocytes, T cells, B cells, natural killer cells, leukocytes, lymphocytes, dendritic cells, and macrophages.

Embodiments include methods of polarizing a macrophage by contacting a macrophage comprising a chimeric receptor with a ligand for the extracellular domain of the chimeric receptor; binding the ligand to the extracellular domain of the chimeric receptor. The binding of the ligand to the extracellular domain of the chimeric receptor activates the cytoplasmic portion and the activation of the cytoplasmic portion polarizes the macrophage.

Nucleotide, polynucleotide, or nucleic acid sequence will be understood according to the present disclosure as meaning both a double-stranded or single-stranded DNA or RNA in the monomeric and dimeric (so-called in tandem) forms and the transcription products of the DNAs or RNAs.

Aspects of the disclosure relate nucleotide sequences which it has been possible to isolate, purify or partially purify, starting from separation methods such as, for example, ion-exchange chromatography, by exclusion based on molecular size, or by affinity, or alternatively fractionation techniques based on solubility in different solvents, or starting from methods of genetic engineering such as amplification, cloning, and subcloning, it being possible for the sequences to be carried by vectors.

A nucleotide sequence fragment will be understood as designating any nucleotide fragment, and may include, by way of non-limiting examples, length of at least 8, 12, 20, 25, 50, 75, 100, 200, 300, 400, 500, 1000, or more, consecutive nucleotides of the sequence from which it originates.

A specific fragment of a nucleotide sequence will be understood as designating any nucleotide fragment of, having, after alignment and comparison with the corresponding wild-type sequence, at least one less nucleotide or base.

Homologous nucleotide sequence as used herein is understood as meaning a nucleotide sequence having at least a percentage identity with the bases of a nucleotide sequence of at least about 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7%, this percentage being purely statistical and it being possible to distribute the differences between the two nucleotide sequences at random and over the whole of their length.

Specific homologous nucleotide sequences in the sense of the present disclosure is understood as meaning a homologous sequence having at least one sequence of a specific fragment, such as defined above. The “specific” homologous sequences can comprise, for example, the sequences corresponding to a genomic sequence or to the sequences of its fragments representative of variants of the genomic sequence. These specific homologous sequences can thus correspond to variations linked to mutations within the sequence and especially correspond to truncations, substitutions, deletions and/or additions of at least one nucleotide. The homologous sequences can likewise correspond to variations linked to the degeneracy of the genetic code.

The term “degree or percentage of sequence homology” refers to “degree or percentage of sequence identity between two sequences after optimal alignment” as defined in the present application.

Two nucleotide sequences are said to be “identical” if the sequence of amino-acids or nucleotidic residues, in the two sequences is the same when aligned for maximum correspondence as described below. Sequence comparisons between two (or more) peptides or polynucleotides are typically performed by comparing sequences of two optimally aligned sequences over a segment or “comparison window” to identify and compare local regions of sequence similarity. Optimal alignment of sequences for comparison may be conducted by the local homology algorithm of Smith and Waterman, Ad. App. Math 2:482 (1981), by the homology alignment algorithm of Neddleman and Wunsch, J. Mol. Biol. 48:443 (1970), by the search for similarity method of Pearson and Lipman, Proc. Natl. Acad. Sci. (U.S.A.) 85:2444 (1988), by computerized implementation of these algorithms (GAP, BESTFIT, FASTA, and TFASTA in the Wisconsin Genetics Software Package, Genetics Computer Group (GCG), 575 Science Dr., Madison, Wis.), or by visual inspection.

“Percentage of sequence identity” (or degree of identity) is determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity.

The definition of sequence identity given above is the definition that would be used by one of skill in the art. The definition by itself does not need the help of any algorithm, the algorithms being helpful only to achieve the optimal alignments of sequences, rather than the calculation of sequence identity.

From the definition given above, it follows that there is a well defined and only one value for the sequence identity between two compared sequences which value corresponds to the value obtained for the best or optimal alignment.

In the BLAST N or BLAST P “BLAST 2 sequence,” software which is available in the web site worldwideweb.ncbi.nlm.nih.gov/gorf/bl2.html, and habitually used by the inventors and in general by the skilled person for comparing and determining the identity between two sequences, gap cost which depends on the sequence length to be compared is directly selected by the software (i.e., 11.2 for substitution matrix BLOSUM-62 for length>85).

Complementary nucleotide sequence of a sequence as used herein is understood as meaning any DNA whose nucleotides are complementary to those of the sequences and whose orientation is reversed (antisense sequence).

Hybridization under conditions of stringency with a nucleotide sequence as used herein is understood as meaning hybridization under conditions of temperature and ionic strength chosen in such a way that they allow the maintenance of the hybridization between two fragments of complementary DNA.

By way of illustration, conditions of great stringency of the hybridization step with the aim of defining the nucleotide fragments described above are advantageously the following.

The hybridization is carried out at a preferential temperature of 65° C. in the presence of SSC buffer, 1×SSC corresponding to 0.15 M NaCl and 0.05 M Na citrate. The washing steps, for example, can be the following: 2×SSC, at ambient temperature followed by two washes with 2×SSC, 0.5% SDS at 65° C.; 2×0.5×SSC, 0.5% SDS; at 65° C. for 10 minutes cach.

The conditions of intermediate stringency, using, for example, a temperature of 42° C. in the presence of a 2×SSC buffer, or of less stringency, for example a temperature of 37° C. in the presence of a 2×SSC buffer, respectively require a globally less significant complementarity for the hybridization between the two sequences.

The stringent hybridization conditions described above for a polynucleotide with a size of approximately 350 bases will be adapted by the person skilled in the art for oligonucleotides of greater or smaller size, according to the teaching of Sambrook et al., 1989.

Among the nucleotide sequences described herein, are those which can be used as a primer or probe in methods allowing the homologous sequences to be obtained, these methods, such as the polymerase chain reaction (PCR), nucleic acid cloning, and sequencing, being well known to the person skilled in the art.

Among the nucleotide sequences are those which can be used as a primer or probe in methods allowing the presence of specific nucleic acids, one of their fragments, or one of their variants such as defined below to be determined. In embodiments, the nucleotide sequences may comprise fragments of SEQ ID NOS: 2, 6, 8, 10, 12, 14, 16, 18, 20, 22, and 24 which encode a transmembrane domain, cytosolic domain, or a portion thereof. Further fragments may include nucleotide sequences encoding linkers, hinges, or fragments thereof such as nucleotides encoding one or more of SEQ ID NOS: 26-34. Further fragments may include fragments of nucleotide sequences encoding one or more of SEQ ID NOS: 35-54.

The nucleotide sequence fragments can be obtained, for example, by specific amplification, such as PCR, or after digestion with appropriate restriction enzymes of nucleotide sequences, these methods in particular being described in the work of Sambrook et al., 1989. Also, such fragments may be obtained with gene synthesis standard technology available from companies such as GENSCRIPT®. Such representative fragments can likewise be obtained by chemical synthesis according to methods well known to persons of ordinary skill in the art.

Modified nucleotide sequence will be understood as meaning any nucleotide sequence obtained by mutagenesis according to techniques well known to the person skilled in the art, and containing modifications with respect to a wild-type sequence, for example mutations in the regulatory and/or promoter sequences of polypeptide expression, especially leading to a modification of the rate of expression of the polypeptide or to a modulation of the replicative cycle.

Modified nucleotide sequence will likewise be understood as meaning any nucleotide sequence coding for a modified polypeptide such as defined below.

Disclosed are nucleotide sequences encoding a chimeric receptor, the nucleotide sequences comprising nucleotide sequences selected from SEQ ID NOS: 2, 6, 8, 10, 12, 14, 16, 18, 20, 22, and 24 or one of their fragments. Such fragments may encode particular domains such as transmembrane domains or cytosolic domains or portions thereof. Further nucleotide sequences encoding a chimeric receptor may include nucleotide sequences encoding linkers, hinges, or fragments thereof such as nucleotides encoding one or more of SEQ ID NOS: 26-34. Nucleotide sequences encoding a chimeric receptor may further nucleotide sequences encoding one or more of SEQ ID NOS: 35-54 or fragments thereof.

Embodiments likewise relate to nucleotide sequences characterized in that they comprise a nucleotide sequence selected from: a) at least one of a nucleotide sequence of SEQ ID NOS: 2, 6, 8, 10, 12, 14, 16, 18, 20, 22, and 24, a nucleotide sequence encoding at least one of SEQ ID NOS: 25-54, or one of their fragments; b) a nucleotide sequence of a specific fragment of a sequence such as defined in a); c) a homologous nucleotide sequence having at least 80% identity with a sequence such as defined in a) or b); d) a complementary nucleotide sequence or sequence of RNA corresponding to a sequence such as defined in a), b) or c); and c) a nucleotide sequence modified by a sequence such as defined in a), b), c) or d).

Among the nucleotide sequences are the nucleotide sequences of SEQ ID NOS: 2, 6, 8, 10, 12, 14, 16, 18, 20, 22, and 24, a nucleotide sequence encoding at least one of SEQ ID NOS: 25-54, or fragments thereof and any nucleotide sequences which have a homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with the at least one of the sequences of SEQ ID NOS: 2, 6, 8, 10, 12, 14, 16, 18, 20, 22, and 24 a nucleotide sequence encoding at least one of SEQ ID NOS: 25-54, or fragments thereof. The homologous sequences can comprise, for example, the sequences corresponding to the wild-type sequences. In the same manner, these specific homologous sequences can correspond to variations linked to mutations within the wild-type sequence and especially correspond to truncations, substitutions, deletions and/or additions of at least one nucleotide. As will be apparent to one of ordinary skill in the art, such homologues are easily created and identified using standard techniques and publicly available computer programs such as BLAST. As such, cach homologue referenced above should be considered as set forth herein and fully described.

Embodiments comprise the chimeric receptors coded for by a nucleotide sequence described herein, or fragments thereof, whose sequence is represented by a fragment. Amino acid sequences corresponding to the polypeptides which can be coded for according to one of the three possible reading frames of at least one of the sequences of SEQ ID NOS: 35-54.

Embodiments likewise relate to chimeric receptors, characterized in that they comprise a polypeptide selected from at least one of the amino acid sequences of SEQ ID NOS: 1, 3, 5, 7, 9, 11, 13, 15, 17, 19, 21, 23, and 25-54, or one of their fragments.

Among the polypeptides, according to embodiments, are the polypeptides of amino acid sequence SEQ ID NOS: 3, 5, 7, 9, 11, 13, 15, 17, 19, 21, 23, and 25-54, or fragments thereof or any other polypeptides which have a homology of at least 80%, 81%, 82%, 83%, 84%, 85%, 86%, 87%, 88%, 89%, 90%, 91%, 92%, 93%, 94%, 95%, 96%, 97%, 98%, 99%, 99.5%, 99.6%, or 99.7% identity with at least one of the sequences of SEQ ID NOS: 1, 3, 5, 7, 9, 11, 13, 15, 17, 19, 21, 23, and 25-54 or fragments thereof. As will be apparent to one of ordinary skill in the art, such homologues are easily created and identified using standard techniques and publicly available computer programs such as BLAST. As such, each homologue referenced above should be considered as set forth herein and fully described.

Embodiments also relate to the polypeptides, characterized in that they comprise a polypeptide selected from: a) a specific fragment of at least 5 amino acids of a polypeptide of an amino acid sequence; b) a polypeptide homologous to a polypeptide such as defined in a); c) a specific biologically active fragment of a polypeptide such as defined in a) or b); and d) a polypeptide modified by a polypeptide such as defined in a), b) or c).

In the present description, the terms polypeptide, peptide and protein are interchangeable.

In embodiments, the chimeric receptors may be glycosylated, pegylated, and/or otherwise post-translationally modified. In further embodiments, glycosylation, pegylation, and/or other posttranslational modifications may occur in vivo or in vitro and/or may be performed using chemical techniques. In additional embodiments, any glycosylation, pegylation and/or other posttranslational modifications may be N-linked or O-linked.

In embodiments any one of the chimeric receptors may be enzymatically or functionally active such that, when the extracellular domain is bound by a ligand, a signal is transduced to polarize a macrophage.

As used herein, a “polarized macrophage” is a macrophage that correlates with an M1 or M2 macrophage phenotype. M1 polarized macrophages secrete IL-12 and IL-23. The determination of a macrophage as polarized to M1 may be performed by measuring the expression of IL-12 and/or IL-23 using a standard cytokine assay and comparing that expression to the expression by newly differentiated unpolarized macrophages. Alternatively, the determination can be made by determining if the cells are CD14+, CD80+, CD206+, and CDCD163−. M2 polarized macrophages secrete IL-10. The determination of a macrophage as polarized to M2 may be performed by measuring the expression of IL-10 using a standard cytokine assay and comparing that expression to the expression by newly differentiated unpolarized macrophages. Alternatively, the determination can be made by determining if the cells are CD14+, CD80−, CD206+, and CDCD163+

Aspects of the disclosure relate to chimeric receptors obtained by genetic recombination, or alternatively by chemical synthesis and that they may thus contain unnatural amino acids, as will be described below.

A “polypeptide fragment” according to the embodiments is understood as designating a polypeptide containing at least 5 consecutive amino acids, preferably 10 consecutive amino acids or 15 consecutive amino acids.

Herein, a specific polypeptide fragment is understood as designating the consecutive polypeptide fragment coded for by a specific fragment a nucleotide sequence.

“Homologous polypeptide” will be understood as designating the polypeptides having, with respect to the natural polypeptide, certain modifications such as, in particular, a deletion, addition, or substitution of at least one amino acid, a truncation, a prolongation, a chimeric fusion, and/or a mutation. Among the homologous polypeptides, those are preferred whose amino acid sequence has at least 80% or 90%, homology with the sequences of amino acids of polypeptides described herein.

“Specific homologous polypeptide” will be understood as designating the homologous polypeptides such as defined above and having a specific fragment of polypeptide polypeptides described herein.

In the case of a substitution, one or more consecutive or nonconsecutive amino acids are replaced by “equivalent” amino acids. The expression “equivalent” amino acid is directed here at designating any amino acid capable of being substituted by one of the amino acids of the base structure without, however, essentially modifying the biological activities of the corresponding peptides and such that they will be defined by the following. As will be apparent to one of ordinary skill in the art, such substitutions are easily created and identified using standard molecular biology techniques and publicly available computer programs such as BLAST. As such, cach substitution referenced above should be considered as set forth herein and fully described.

These equivalent amino acids can be determined either by depending on their structural homology with the amino acids which they substitute, or on results of comparative tests of biological activity between the different polypeptides, which are capable of being carried out.

By way of nonlimiting example, the possibilities of substitutions capable of being carried out without resulting in an extensive modification of the biological activity of the corresponding modified polypeptides will be mentioned, the replacement, for example, of leucine by valine or isoleucine, of aspartic acid by glutamic acid, of glutamine by asparagine, of arginine by lysine etc., the reverse substitutions naturally being envisageable under the same conditions.

In a further embodiment, substitutions are limited to substitutions in amino acids not conserved among other proteins which have similar identified enzymatic activity. For example, one of ordinary skill in the art may align proteins of the same function in similar organisms and determine which amino acids are generally conserved among proteins of that function. One example of a program that may be used to generate such alignments is wordlwideweb.charite.de/bioinf/strap/in conjunction with the databases provided by the NCBI.

Thus, according to one embodiment, substitutions or mutation may be made at positions that are generally conserved among proteins of that function. In a further embodiment, nucleic acid sequences may be mutated or substituted such that the amino acid they code for is unchanged (degenerate substitutions and/mutations) and/or mutated or substituted such that any resulting amino acid substitutions or mutation are made at positions that are generally conserved among proteins of that function.

The specific homologous polypeptides likewise correspond to polypeptides coded for by the specific homologous nucleotide sequences such as defined above and thus comprise in the present definition the polypeptides which are mutated or correspond to variants which can exist in wild-type sequences, and which especially correspond to truncations, substitutions, deletions, and/or additions of at least one amino acid residue.

“Specific biologically active fragment of a polypeptide” as used herein will be understood in particular as designating a specific polypeptide fragment, such as defined above, having at least one of the characteristics of polypeptides described herein. In certain embodiments the peptide is capable of behaving as chimeric antigen receptor that when activated polarizes a macrophage.

“Modified polypeptide” of a polypeptide as used herein is understood as designating a polypeptide obtained by genetic recombination or by chemical synthesis as will be described below, having at least one modification with respect to a wild-type sequence. These modifications may or may not be able to bear on amino acids at the origin of specificity, and/or of activity, or at the origin of the structural conformation, localization, and of the capacity of membrane insertion of the polypeptide as described herein. It will thus be possible to create polypeptides of equivalent, increased, or decreased activity, and of equivalent, narrower, or wider specificity. Among the modified polypeptides, it is necessary to mention the polypeptides in which up to 5 or more amino acids can be modified, truncated at the N-or C-terminal end, or even deleted or added.

The methods allowing the modulations on eukaryotic or prokaryotic cells to be demonstrated are well known to the person of ordinary skill in the art. It is likewise well understood that it will be possible to use the nucleotide sequences coding for the modified polypeptides for the modulations, for example through vectors and described below.

The preceding modified polypeptides can be obtained by using combinatorial chemistry, in which it is possible to systematically vary parts of the polypeptide before testing them on models, cell cultures or microorganisms for example, to select the compounds which are most active or have the properties sought.

Chemical synthesis likewise has the advantage of being able to use unnatural amino acids, or nonpeptide bonds.

Thus, in order to improve the duration of life of the polypeptides, it may be of interest to use unnatural amino acids, for example in D form, or else amino acid analogs, especially sulfur-containing forms, for example.

Finally, it will be possible to integrate the structure of the polypeptides, its specific or modified homologous forms, into chemical structures of polypeptide type or others. Thus, it may be of interest to provide at the N-and C-terminal ends compounds not recognized by proteases.

The nucleotide sequences coding for a polypeptide are likewise disclosed herein.

Embodiments likewise relates to nucleotide sequences utilizable as a primer or probe, characterized in that the sequences are selected from the nucleotide sequences described herein.

It is well understood that various embodiments likewise relate to specific polypeptides including chimeric receptors, coded for by nucleotide sequences, capable of being obtained by purification from natural polypeptides, by genetic recombination or by chemical synthesis by procedures well known to the person skilled in the art and such as described in particular below. In the same manner, the labeled or unlabeled mono-or polyclonal antibodies directed against the specific polypeptides coded for by the nucleotide sequences are also encompassed by this disclosure.

Embodiments additionally relate to the use of a nucleotide sequence as a primer or probe for the detection and/or the amplification of nucleic acid sequences.

The nucleotide sequences according to embodiments can thus be used to amplify nucleotide sequences, especially by the PCR technique (polymerase chain reaction) (Erlich, 1989; Innis et al., 1990; Rolfs et al., 1991; and White et al., 1997).

These oligodeoxyribonucleotide or oligoribonucleotide primers advantageously have a length of at least 8 nucleotides, preferably of at least 12 nucleotides, and even more preferentially at least 20 nucleotides.

Other amplification techniques of the target nucleic acid can be advantageously employed as alternatives to PCR.

The nucleotide sequences described herein, in particular the primers, can likewise be employed in other procedures of amplification of a target nucleic acid, such as: the TAS technique (Transcription-based Amplification System), described by Kwoh et al. in 1989; the 3SR technique (Self-Sustained Sequence Replication), described by Guatelli et al. in 1990; the NASBA technique (Nucleic Acid Sequence Based Amplification), described by Kievitis et al. in 1991; the SDA technique (Strand Displacement Amplification) (Walker et al., 1992); the TMA technique (Transcription Mediated Amplification).

The polynucleotides, including chimeric receptors, can also be employed in techniques of amplification or of modification of the nucleic acid serving as a probe, such as: the LCR technique (Ligase Chain Reaction), described by Landegren et al. in 1988 and improved by Barany et al. in 1991, which employs a thermostable ligase; the RCR technique (Repair Chain Reaction), described by Segev in 1992; the CPR technique (Cycling Probe Reaction), described by Duck et al. in 1990; the amplification technique with Q-beta replicase, described by Miele et al. in 1983 and especially improved by Chu et al. in 1986, Lizardi et al. in 1988, then by Burg et al. as well as by Stone et al. in 1996.

In the case where the target polynucleotide to be detected is possibly an RNA, for example an mRNA, it will be possible to use, prior to the employment of an amplification reaction with the aid of at least one primer or to the employment of a detection procedure with the aid of at least one probe, an enzyme of reverse transcriptase type in order to obtain a cDNA from the RNA contained in the biological sample. The cDNA obtained will thus serve as a target for the primer(s) or the probe(s) employed in the amplification or detection procedure.

The detection probe will be chosen in such a manner that it hybridizes with the target sequence or the amplicon generated from the target sequence. By way of sequence, such a probe will advantageously have a sequence of at least 12 nucleotides, in particular of at least 20 nucleotides, and preferably of at least 100 nucleotides.

Embodiments also comprise the nucleotide sequences utilizable as a probe or primer, characterized in that they are labeled with a radioactive compound or with a nonradioactive compound.

The unlabeled nucleotide sequences can be used directly as probes or primers, although the sequences are generally labeled with a radioactive isotope (32p, 35S, 3H, 1251) or with a nonradioactive molecule (biotin, acetylaminofluorene, digoxigenin, 5-bromodeoxyuridine, fluorescein) to obtain probes which are utilizable for numerous applications.

Examples of nonradioactive labeling of nucleotide sequences are described, for example, in French Patent No. 78.10975 or by Urdea et al. or by Sanchez-Pescador et al. in 1988.

In the latter case, it will also be possible to use one of the labeling methods described in patents FR-2 422 956 and FR-2 518 755.

The hybridization technique can be carried out in various manners (Matthews et al., 1988). The most general method consists in immobilizing the nucleic acid extract of cells on a support (such as nitrocellulose, nylon, polystyrene) and in incubating, under well-defined conditions, the immobilized target nucleic acid with the probe. After hybridization, the excess of probe is eliminated and the hybrid molecules formed are detected by the appropriate method (measurement of the radioactivity, of the fluorescence or of the enzymatic activity linked to the probe).

Various embodiments likewise comprise the nucleotide sequences or polypeptide sequences described herein, characterized in that they are immobilized on a support, covalently or noncovalently.

According to another advantageous mode of employing nucleotide sequences, the latter can be used immobilized on a support and can thus serve to capture, by specific hybridization, the target nucleic acid obtained from the biological sample to be tested. If necessary, the solid support is separated from the sample and the hybridization complex formed between the capture probe and the target nucleic acid is then detected with the aid of a second probe, a so-called detection probe, labeled with an easily detectable element.

Another aspect is a vector for the cloning and/or expression of a sequence, characterized in that it contains a nucleotide sequence described herein.

The vectors, characterized in that they contain the elements allowing the integration, expression and/or the secretion of the nucleotide sequences in a determined host cell, are likewise provided.

The vector may then contain a promoter, signals of initiation and termination of translation, as well as appropriate regions of regulation of transcription. It may be able to be maintained stably in the host cell and can optionally have particular signals specifying the secretion of the translated protein. These different elements may be chosen as a function of the host cell used. To this end, the nucleotide sequences described herein may be inserted into autonomous replication vectors within the chosen host, or integrated vectors of the chosen host.

Such vectors will be prepared according to the methods currently used by the person skilled in the art, and it will be possible to introduce the clones resulting therefrom into an appropriate host by standard methods, such as, for example, calcium phosphate precipitation, lipofection, electroporation, and thermal shock.

The vectors according are, for example, vectors of plasmid or viral origin. Examples of vectors for the expression of polypeptides described herein are plasmids, phages, cosmids, artificial chromosomes, viral vectors, AAV vectors, baculovirus vectors, adenoviral vectors, lentiviral vectors, retroviral vectors, chimeric viral vectors, and chimeric adenoviridae such as AD5/F35.

These vectors are useful for transforming host cells in order to clone or to express the nucleotide sequences described herein.

Embodiments likewise comprise the host cells transformed by a vector.

These cells can be obtained by the introduction into host cells of a nucleotide sequence inserted into a vector such as defined above, then the culturing of the cells under conditions allowing the replication and/or expression of the transfected nucleotide sequence.

The host cell can be selected from prokaryotic or eukaryotic systems, such as, for example, bacterial cells (Olins and Lee, 1993), but likewise yeast cells (Buckholz, 1993), as well as plants cells, such as Arabidopsis sp., and animal cells, in particular the cultures of mammalian cells (Edwards and Aruffo, 1993), for example, HEK 293, cells, HEK 293T cells, Chinese hamster ovary (CHO) cells, myeloid cells, myeloid progenitor cells, monocytes, neutrophils, basophils, cosinophils, megakaryocytes, T cells, B cells, natural killer cells, leukocytes, lymphocytes, dendritic cells, and macrophages, but likewise the cells of insects in which it is possible to use procedures employing baculoviruses, for example sf9 insect cells (Luckow, 1993).

Embodiments likewise relate to organisms comprising one of the transformed cells.

The obtainment of transgenic organisms expressing one or more of the nucleic acids or part of the nucleic acids may be carried out in, for example, rats, mice, or rabbits according to methods well known to the person skilled in the art, such as by viral or nonviral transfections. It will be possible to obtain the transgenic organisms expressing one or more of the genes by transfection of multiple copies of the genes under the control of a strong promoter of ubiquitous nature, or selective for one type of tissue. It will likewise be possible to obtain the transgenic organisms by homologous recombination in embryonic cell strains, transfer of these cell strains to embryos, selection of the affected chimeras at the level of the reproductive lines, and growth of the chimeras.

The transformed cells as well as the transgenic organisms are utilizable in procedures for preparation of recombinant polypeptides.

It is today possible to produce recombinant polypeptides in relatively large quantity by genetic engineering using the cells transformed by expression vectors or using transgenic organisms.

The procedures for preparation of a polypeptide, such as a chimeric receptor, in recombinant form, characterized in that they employ a vector and/or a cell transformed by a vector and/or a transgenic organism comprising one of the transformed cells are themselves comprised in in the present disclosure.

As used herein, “transformation” and “transformed” relate to the introduction of nucleic acids into a cell, whether prokaryotic or eukaryotic. Further, “transformation” and “transformed,” as used herein, need not relate to growth control or growth deregulation.

Among the procedures for preparation of a polypeptide, such as a chimeric receptor, in recombinant form, the preparation procedures employing a vector, and/or a cell transformed by the vector and/or a transgenic organism comprising one of the transformed cells, containing a nucleotide sequence, such as those encoding a chimeric receptor.

A variant according, as used herein, may consist of producing a recombinant polypeptide fused to a “carrier” protein (chimeric protein). The advantage of this system is that it may allow stabilization of and/or a decrease in the proteolysis of the recombinant product, an increase in the solubility in the course of renaturation in vitro and/or a simplification of the purification when the fusion partner has an affinity for a specific ligand.

More particularly, embodiments relate to a procedure for preparation of a polypeptide comprising the following steps: a) culture of transformed cells under conditions allowing the expression of a recombinant polypeptide of nucleotide sequence; b) if need be, recovery of the recombinant polypeptide.

When the procedure for preparation of a polypeptide, such as a chimeric receptor, employs a transgenic organism, the recombinant polypeptide may then extracted from the organism or left in place.

Embodiments also relate to a polypeptide which is capable of being obtained by a procedure such as described previously.

Embodiments also comprise a procedure for preparation of a synthetic polypeptide, characterized in that it uses a sequence of amino acids of polypeptides.

This disclosure likewise relates to a synthetic polypeptide, such as a chimeric receptor, obtained by a procedure.

The polypeptides, such as chimeric receptors, can likewise be prepared by techniques which are conventional in the field of the synthesis of peptides. This synthesis can be carried out in homogeneous solution or in solid phase.

For example, recourse can be made to the technique of synthesis in homogeneous solution described by Houben-Weyl in 1974.

This method of synthesis consists in successively condensing, two by two, the successive amino acids in the order required, or in condensing amino acids and fragments formed previously and already containing several amino acids in the appropriate order, or alternatively several fragments previously prepared in this way, it being understood that it will be necessary to protect beforehand all the reactive functions carried by these amino acids or fragments, with the exception of amine functions of one and carboxyls of the other or vice-versa, which must normally be involved in the formation of peptide bonds, especially after activation of the carboxyl function, according to the methods well known in the synthesis of peptides.

Recourse may also be made to the technique described by Merrifield.

To make a peptide chain according to the Merrifield procedure, recourse is made to a very porous polymeric resin, on which is immobilized the first C-terminal amino acid of the chain. This amino acid is immobilized on a resin through its carboxyl group and its amine function is protected. The amino acids which are going to form the peptide chain are thus immobilized, one after the other, on the amino group, which is deprotected beforehand each time, of the portion of the peptide chain already formed, and which is attached to the resin. When the whole of the desired peptide chain has been formed, the protective groups of the different amino acids forming the peptide chain are eliminated and the peptide is detached from the resin with the aid of an acid.

These hybrid molecules can be formed, in part, of a polypeptide carrier molecule or of fragments thereof, associated with a possibly immunogenic part, in particular an epitope of the diphtheria toxin, the tetanus toxin, a surface antigen of the hepatitis B virus (patent FR 79 21811), the VPI antigen of the poliomyelitis virus or any other viral or bacterial toxin or antigen.

The polypeptides, including chimeric receptors, the antibodies described below and the nucleotide sequences encoding any of the foregoing can advantageously be employed in procedures for the polarization of a macrophage.

In embodiments, a nucleic acid sequence encoding a chimeric receptor is provided to a cell. The cell may then express the encoded chimeric receptor. The expressed chimeric receptor may be present on the surface of the cell or in the cytoplasm. In particular embodiments, the cell expressing the chimeric receptor is a macrophage. The macrophage expressed chimeric receptor may bind a ligand, and binding of the ligand may activate the chimeric receptor so as to induce polarization of the macrophage as previously described.

In embodiments, the cell provided with the nucleic acid sequence encoding a chimeric receptor may be isolated from a subject. After the cell is provided with the nucleic acid, the cell may be returned to the subject from whom it was obtained, for example by injection or transfusion. In other embodiments, the cell provided with the nucleic acid may be provided by a donor. After the donor cell is provided with the nucleic acid, the cell may then be provided to an individual other than the donor. Examples of donor cells include, but are not limited to primary cells from a subject and cells from a cell line.

In other embodiments, chimeric receptors may be introduced directly into cells. Any method of introducing a protein into cell may be used, including, but not limited to, microinjection, electroporation, membrane fusion, and the use of protein transduction domains. After the cell is provided with chimeric receptors, the cell may be returned to the subject from whom it was obtained, for example by injection or transfusion. In other embodiments, the cell provided with the chimeric receptors is provided by a donor. After the donor cell is provided with the nucleic acid, the cell may then be provided to an individual other than the donor. Examples of donor cells include, but are not limited to primary cells from a subject and cells from a cell line.

Embodiments likewise relates to polypeptides, such as chimeric receptors, labeled with the aid of an adequate label, such as, of the enzymatic, fluorescent or radioactive type.

The polypeptides allow monoclonal or polyclonal antibodies to be prepared which are characterized in that they specifically recognize the polypeptide. It will advantageously be possible to prepare the monoclonal antibodies from hybridomas according to the technique described by Kohler and Milstein in 1975. It will be possible to prepare the polyclonal antibodies, for example, by immunization of an animal, in particular a mouse, with a polypeptide or a DNA, associated with an adjuvant of the immune response, and then purification of the specific antibodies contained in the scrum of the immunized animals on an affinity column on which the polypeptide which has served as an antigen has previously been immobilized. The polyclonal antibodies can also be prepared by purification, on an affinity column on which a polypeptide has previously been immobilized, of the antibodies contained in the serum of an animal immunologically challenged by a chimeric receptor, or a polypeptide or fragment thereof.

In addition, antibodies can be used to prepare other forms of binding molecules, including, but not limited to, IgAs, IgDs, IgEs, IgGs, IgMs, Fab fragments, F(ab′)₂ fragments, monovalent antibodies, scFv fragments, scRv-Fc fragments, IgNARs, hclgGs, VhH antibodies, nanobodies, and alphabodies.

Embodiments likewise relates to mono-or polyclonal antibodies or their fragments, or chimeric antibodies, or fragments thereof, characterized in that they are capable of specifically recognizing a polypeptide described herein or a ligand of a polypeptide and/or chimeric receptor.

It will likewise be possible for the antibodies to be labeled in the same manner as described previously for the nucleic probes, such as a labeling of enzymatic, fluorescent or radioactive type. It will be also be possible to include such antibodies and/or fragments thereof as part of a chimeric receptor. By way of non-limiting example, such an antibody or fragment thereof may make up a portion of the extracellular domain of a chimeric receptor.

Embodiments are additionally directed at a procedure for the detection and/or identification of chimeric receptor in a sample, characterized in that it comprises the following steps: a) contacting of the sample with a mono-or polyclonal (under conditions allowing an immunological reaction between the antibodies and the chimeric receptor possibly present in the biological sample); b) demonstration of the antigen-antibody complex possibly formed.

The embodiments are described in additional detail in the following illustrative examples. Although the examples may represent only selected embodiments, it should be understood that the following examples are illustrative and not limiting.

### EXAMPLES

### Example 1

### Isolation of ScFv Fragments for Specific Ligands

cDNA was purified from a monoclonal antibody hybridoma cell (CB1) expressing an antibody specific to human TK1. The isolated cDNA was used to amplify the heavy and light chains of the CB1 variable region via polymerase chain reaction (PCR) Sequences from the heavy and light chain were confirmed using NCBI Blast. CB1 heavy and light chains were fused together via site overlap extension (SOE) PCR to form a single chain fragment variable (scFv) using a G4S linker. The G4S linker was codon optimized for yeast and humans using the Codon Optimization tool provided by IDT (https://www.idtdna.com/CodonOpt) in order to maximize protein expression. The CB1 scFv was cut out using restriction enzymes and inserted into a pMP71 CAR vector.

TK-1 and HPRT-specific human scFv fragments were isolated from a yeast antibody library. TK-1 and HPRT proteins were isolated, His-tagged, and purified. TK-1 and HPRT protein were labeled with an anti-His biotinylated antibody and added to the library to select for TK-1 and HPRT-specific antibody clones. TK-1 and HPRT antibody clones were alternately stained with streptavidin or anti-biotin microbeads and enriched using a magnetic column. Two additional rounds of sorting and selection were performed to isolate TK-1 and HPRT specific antibodies. For the final selection, possible TK-1 and HPRT antibody clones and their respective proteins were sorted by fluorescence-activated cell sorting (FACS) by alternately labeling with fluorescently-conjugated anti-HA or anti-c-myc antibodies to isolate TK-1 and HPRT specific antibodies. High affinity clones were selected for chimeric receptor construction. Other human antibodies or humanized antibodies from other animals could be selected or altered to be TK-1 or HPRT specific by using phage display or other recombination methods.

Selected scFv clones were then combined with human IgGI constant domains to create an antibody for use in applications such as Western blot or ELISA in order to confirm the binding specificity of the scFv. The antibody construct was inserted into the pPNL9 yeast secretion vector and YVH10 yeast were transformed with the construct and induced to produce the antibody. Other expression systems such as E. coli or mammalian systems could also be used to secrete antibodies.

Isolation and characterization of protein-specific antibody fragments.

Referring to FIG. 26, 105 yeast were incubated with 2.5 μg of protein of interest labeled with the fluorescent tag APC. The higher left (red) peak indicates yeast population that was not binding to the protein of interest (negative control). The lower left (blue) peak on the left illustrates yeast not expressing their surface protein while the high (blue) peak on the right indicates binding of the expressed antibody fragment to the protein of interest.

Structural Consensus among Antibodies Defines the Antigen 5 Binding Site, PLOS Comput. Biol. 8 (2): e1002388. doi: 10.1371/journal.pcbi.1002388, Kunik V, Ashkenazi S, Ofran Y (2012). Paratome: An online tool for systematic identification of antigen binding regions in antibodies based on sequence or structure, Nucleic Acids Res. 2012 July; 40 (Web Server issue): W521-4. doi: 10.1093/nar/gks480. Epub 2012 Jun. 6.

### Example 2

### Creation of Chimeric Receptors

Construction of chimeric receptor vectors:

The first step in the process is the design of the nucleotide sequences for synthetic chimeric receptor genes and the selection of appropriate lentiviral vectors. All the vector design are carried out in genious software version 9.1.6. The sequences are retrieved from the Uniprot and the Human Protein Reference Data base and NCBI as well.

Vectors are synthesized with a combination of recombinant DNA techniques and gene synthesis.

Sequences for the Single chain variable fragments are produced with a humanized antibody yeast display library or a phage display library. Nucleic acids encoding ScFv specific for each of TK1, HPRT, ROR1, MUC-16, EGFRVIII, Mesothelin, HER2, CEA, BCMA, GPC3, FAP, EphA2, NKG2D ligands, GD2, CD19, CD20, CD30, CD33, CD123, CD133, CD138, and CD171. All possible combinations of nucleic acids encoding chimeric receptors having at least one of each of a), b), c), d), and c), wherein a), b), c), d), and c) are:

a) an ScFv specific for TK1, HPRT, ROR1, MUC-16, EGFRvIII, Mesothelin, HER2, CEA, BCMA, GPC3, FAP, EphA2, NKG2D ligands, GD2, CD19, CD20, CD30, CD33, CD123, CD133, CD138, and CD171;

b) a GS linker or no GS linker;

c) A hinge region selected from an LRR 5 amino acid short hinge, a LRR long hinge, an IgG4 short hinge, an IgG 119 amino acid medium hinge, and IgG4 long hinge, a CD8 hinge, a CD8 hinge with cysteines converted to serines, and no hinge;

d) a transmembrane domain selected from the transmembrane domains of MYD88, TLR3, TLR4, TLR7, TLR8, TLR9, MAL, IRAK1, FCGR2A, FCGR3A, and FCER1G; and

c) a cytosolic domain selected from the cytosolic domains of MYD88,TLR3, TLR4, TLR7, TLR8, TLR9, MAL, IRAK1, FCGR2A, FCGR3A, and FCER1G.

The foregoing nucleic acids encoding chimeric receptors are synthesized with a combination of recombinant DNA techniques and gene synthesis.

Macrophages are genetically modified with an integrated gene delivery method via lentiviral-mediated gene transfer to provide the nucleic acids encoding chimeric receptors. A third generation lentiviral system from addgene is used to package our lentiviral vectors. pHIV-dTomato (#21374) and pUltra-chilli (#48687) are the gene transfer plasmids. pCMV-VSV-G (#8454), pMDLg/pRRE (#12251), pRSV-Rev (#12253), pHCMV-AmphoEnv (#15799) are the packaging plasmids. A lentiviral mediated gene transfer of human lymphocytes has been standardized previously getting efficiencies up to 50% transduction. HEK293T cells are transfected with the calcium phosphate method (SIGMA CAPHOS). Around 10 μg of each packaging plasmid and 20 μg of vector encoding the chimeric receptor are used per transfection. After 48-36 hours viral particles are harvested and sterile filtered. Viral titration are determined infecting HT1080 and U937 cells.

The analysis is performed by flow cytometry detecting a red fluorescent protein. After viral titration human monocytes are transduced using retronectin plates (Clonetech, T100B) and the spin infection method

Previous to lentiviral transduction, monocytes are isolated from whole PBMNCs by negative selection and magnetic sorting using the Monocyte Isolation Kit II, human (MACS130-091-153). After monocyte isolation cells are split in 2 nunclon 6-well plates (Thermo, 145380) seeding 1.5X106 cells in each well for each vector. One plate is immediately transduced while the second plate is used for ex-vivo differentiation of monocytes to M1 macrophages. The M1 macrophages are produced using the media M1-Macrophage Generation Medium DFX (Promocell, C-28055). After 7 days, macrophages are transduced and activated at day 9 with LPS (500X) (Affimetryx, 00-4976-03) and IFN-γ(Promokine, C-60724). The transduction efficiency is analyzed by flow cytometry. Transduced cells are separated by cell sorting using a FACS Aria cell sorter. After cell sorting transduced monocytes are ex vivo cultured for a couple of days before differentiation while differentiated macrophages can last a month.

### Example 3

### Polarization of Macrophages through Chimeric Receptors

The transduced macrophages prepared in Example 2 are separately exposed to TK1, HPRT, ROR1, MUC-16, EGFRVIII, Mesothelin, HER2, CEA, BCMA, GPC3, FAP, EphA2, NKG2D conjugated ligands, GD2, CD19, CD20, CD30, CD33, CD123, CD133, CD138, and CD171and tested for polarization to the M1 phenotype by monitoring the secretion of IL-12 and IL-23 using a standard cytokine assay or by measuring RNA production. Macrophages bearing chimeric receptors are polarized to the M1 phenotype when exposed to the ligand specific for the particular chimeric receptor and determined by increased secretion of IL-12 and/or IL-23. Ligands other than the specific ligand for the specific chimeric receptor display no increase in IL-12 and/or IL-21.

### Example 4

### Production of Monocyte-Derived Macrophages and Transduction

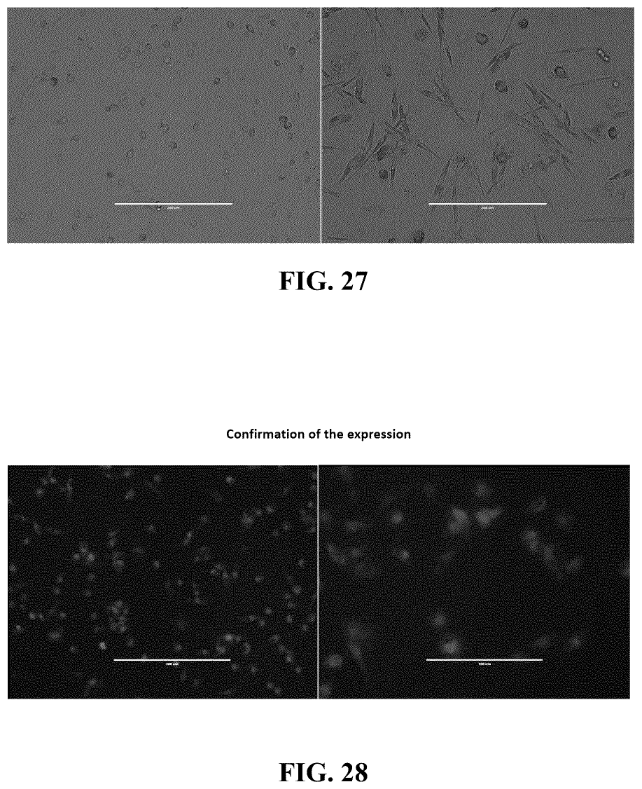

After 7 days of differentiation monocyte-derived macrophages had undergone phenotype changes. These changes where compared between transduced and non-transduced cells. As can be observed in FIG. 27, transduced cells have a more aggressive phenotype similar to M1 or classically activated macrophages. FIG. 27 shows images of Non-transduced and transduced monocyte-derived macrophages at day 8 of differentiation. No Interferon gamma and LPS was added at this point. It can be observed that the phenotype of macrophages transduced with a chimeric receptor is different from non-transduced macrophages. Transduced cells displayed a classically activated or M1-like phenotype indicating macrophage activation. The altered phenotype may be a combined effect of the transduction process and the expression of the new synthetic receptor.

FIG. 28 provides confirmation of the insertion and expression of constructs encoding a chimeric receptor as was confirmed by the expression of dTomato 48-72 hours after transduction. This demonstrates the successful transduction of human monocyte-derived macrophages.

### Example 5

### Transduction Efficiency

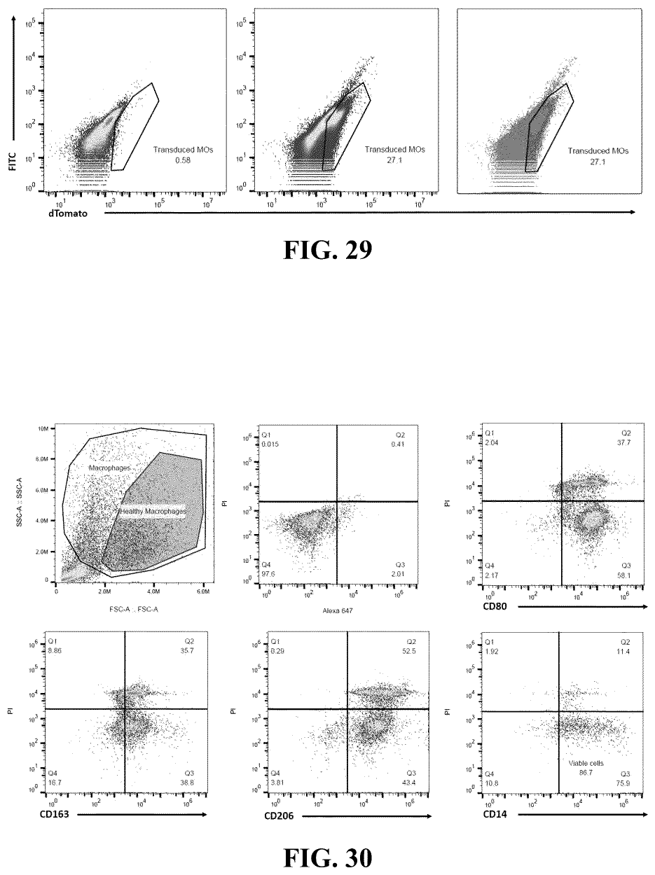

After day 10 of differentiation the transduction efficiency was assessed and macrophages expressing the chimeric receptor were cell sorted. Lentiviral transduction is challenging in macrophages. However, using HIV-1 based systems with EFI-a promoters almost 30% macrophage transduction was achieved. Transductions of the cells at early stages of macrophage differentiation displayed different transduction efficiencies. Monocytes or macrophages in earlier stages of differentiation are easier to transduce. Adenoviral transduction with the chimeric adenovirus AD5/F35 has emerged as another alternative for macrophage transduction. FIG. 29 shows the results of macrophages that were transduced being cell sorted using a FACSAria system. Around 30% of macrophage transduction was achieved using the lentiviral approach. The left most plot shows a control wherein only 0.58% of cells show fluorescence which would indicate expression of dTomato. The right two plots show a transduction efficiency of 27.1 percent after transduction.

### Example 6

### Immunophenotyping of Transduced Macrophages

Immunophenotyping of macrophages transduced with vectors for the expression of a chimeric receptor was performed to identify the activation state of the transduced cells. It has been reported that modifications of the extracellular domain of TLR-4 may induce constant activation of its signaling domain (Gay et al., 2014). Constant activation of the TLR-4 signaling could lead to macrophage activation or M1 phenotype. It is not know if the construct which was used, which is based on TLR-4, is able to trigger a constant activation of the signaling through the TIR domain taken from TLR-4. However, after the transduction process, a change in the phenotype was observed and a change in the expression of cell surface markers in the macrophages. This is likely due to a combination of the lentiviral transduction and the expression of the chimeric receptor protein. Expression of CD14, CD80, D206 and low expression of CD163 were indicators of macrophage polarization towards the M1 phenotype. The expression of these cell surface markers in was observed in the transduced cells. FIG. 30 presents six scatter plots of fluorescence activated cell sorting demonstrating the retention of dye (Alexa 647), and the expression of CD80, CD163, CD206, and CD14 in macrophages transduced with a chimeric receptor.

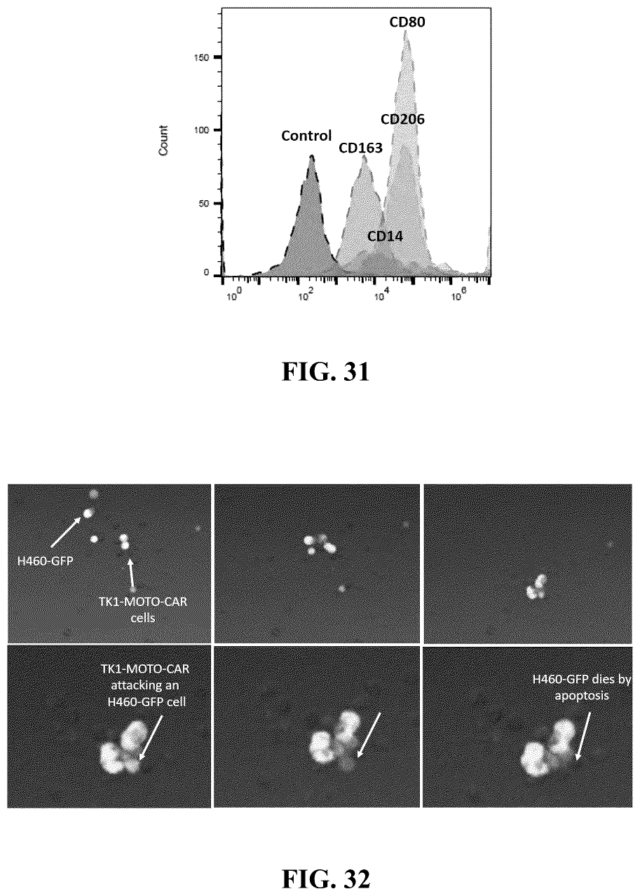

FIG. 31. Presents a histogram of the relative expression levels of M1 cells surface markers in macrophages transduced with a vector to express a chimeric receptor.

### Example 7

### In-vitro Toxicity of TK1 Targeting Chimeric Receptor Transduced Macrophages Against NCI-H460 cells.

The tumoricidal activity of TK1 targeting chimeric receptor transduced macrophages was tested against NCI-H460-GFP cells. The E:T ratio used was 1:10. The analysis was performed with confocal microscopy. Detection of fluorescence was performed every 5 minutes during a 12 hour period. It was observed during time lapse that TK1 targeting chimeric receptor transduced macrophages migrate toward H460-GFP cells and attack them. After the synapsis, specific cell death is induced in the target cell. As demonstrated by the images in FIG. 32, TK1 targeting chimeric receptor transduced macrophages can detect, attack and induce cell death in lung cancer cell lines expressing TK1. NCI-H460 cells were modified to express GFP. The tumoricidal activity of TK1 targeting chimeric receptor transduced macrophages was detected with confocal microscopy as a loss of fluorescence in the target cell.

The disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims and their legal equivalents.

### TABLE OF REFERENCES

1. Hanahan, D., & Weinberg, R. a. (2011). Hallmarks of cancer: the next generation. Cell, 144 (5), 646-74. http://doi.org/10.1016/j.cell.2011.02.013
2. American Cancer Society. (2015). Cancer Facts & Figures 2015.
3. Hoyert, D. L., & Xu, J. (2012). National Vital Statistics Reports Deaths: Preliminary Data for 2011 (Vol. 61).
4. Kurahara, H., Shinchi, H., Mataki, Y., Maemura, K., Noma, H., Kubo, F., . . . Takao, S. (2011). Significance of M2-polarized tumor-associated macrophage in pancreatic cancer. The Journal of Surgical Research, 167 (2), e211-9. http://doi.org/10.1016/j.jss.2009.05.026
5. Steidl, C., Lee, T., & Shah, S. (2010a). Tumor-associated macrophages and survival in classic Hodgkin's lymphoma. The New England Journal of Medicine, 875-885. Retrieved from http://www.nejm.org/doi/full/10.1056/NEJMoa0905680
6. Eiró, N., & Vizoso, F. J. (2012). Inflammation and cancer. World Journal of Gastrointestinal Surgery, 4 (3), 62-72. http://doi.org/10.4240/wjgs.v4.i3.62
7. Kelly, P. M., Davison, R. S., Bliss, E., & McGee, J. O. (1988). Macrophages in human breast disease: a quantitative immunohistochemical study. British Journal of Cancer, 57 (2), 174-7. Retrieved from http://www.pubmedcentral.nih.gov/articlerender.fcgi?artid=2246436&tool=pmcentrez&r endertype=abstract
8. Lewis, C., & Leek, R. (1995). Cytokine regulation of angiogenesis in breast cancer: the role of tumor-associated macrophages. Journal of Leukocyte . . . , 57 (May), 747-751. Retrieved from http://www.jleukbio.org/content/57/5/747.short
9. Mantovani, A., Biswas, S. K., Galdiero, M. R., Sica, A., & Locati, M. (2013). Macrophage plasticity and polarization in tissue repair and remodelling. The Journal of Pathology, 229 (2), 176-85. http://doi.org/10.1002/path.4133
10. Porta, C., Rimoldi, M., Raes, G., Brys, L., Ghezzi, P., Di Liberto, D., . . . Sica, A. (2009). Tolerance and M2 (alternative) macrophage polarization are related processes orchestrated by p50 nuclear factor kappaB. Proceedings of the National Academy of Sciences of the United States of America, 106 (35), 14978-83. http://doi.org/10.1073/pnas.0809784106
11. Sica, A., & Mantovani, A. (2012). Macrophage plasticity and polarization: in vivo veritas. The Journal of Clinical Investigation, 122 (3), 787-796. http://doi.org/10.1172/JCI59643DS1
12. Anderson, C. F., & Mosser, D. M. (2002). A novel phenotype for an activated macrophage: the type 2 activated macrophage. Journal of Leukocyte Biology, 72 (1), 101-6. Retrieved from http://www.ncbi.nlm.nih.gov/pubmed/12101268
13. Ghassabeh, G. H., De Baetselier, P., Brys, L., Noël, W., Van Ginderachter, J. a, Meerschaut, S., . . . Raes, G. (2006). Identification of a common gene signature for type II cytokine-associated myeloid cells elicited in vivo in different pathologic conditions. Blood, 108 (2), 575-83. http://doi.org/10.1182/blood-2005-04-1485
14. Liao, X., Sharma, N., & Kapadia, F. (2011). Krüppel-like factor 4 regulates macrophage polarization. The Journal of Clinical Investigation, 121 (7). http://doi.org/10.1172/JCI45444DS1
15. Davis, M. J., Tsang, T. M., Qiu, Y., Dayrit, J. K., Freij, J. B., Huffnagle, G. B., & Olszewski, M. A. (2013). Macrophage M1/M2 polarization dynamically adapts to changes in cytokine microenvironments in Cryptococcus neoformans infection. mBio, 4 (3), c00264-13. http://doi.org/10.1128/mBio.00264-13
16. Mantovani, A., Sozzani, S., Locati, M., Allavena, P., & Sica, A. (2002). Macrophage polarization: tumor-associated macrophages as a paradigm for polarized M2 mononuclear phagocytes. Trends in Immunology, 23 (11), 549-55. Retrieved from http://www.ncbi.nlm.nih.gov/pubmed/12401408
17. Edin, S., Wikberg, M. L., Dahlin, A. M., Rutegård, J., Oberg, A., Oldenborg, P.-A., & Palmqvist, R. (2012). The distribution of macrophages with a m1 or m2 phenotype in relation to prognosis and the molecular characteristics of colorectal cancer. PloS One, 7 (10), e47045.http://doi.org/10.1371/journal.pone.0047045
18. Forssell, J., Oberg, A., Henriksson, M. L., Stenling, R., Jung, A., & Palmqvist, R. (2007). High macrophage infiltration along the tumor front correlates with improved survival in colon cancer. Clinical Cancer Research, 13 (5), 1472-9. http://doi.org/10.1158/1078-0432.CCR-06-2073
19. Guiducci, C., Vicari, A. P., Sangaletti, S., Trinchieri, G., & Colombo, M. P. (2005). Redirecting in vivo elicited tumor infiltrating macrophages and dendritic cells towards tumor rejection. Cancer Research, 65 (8), 3437-46. http://doi.org/10.1158/0008-5472.CAN-04-4262
20. Baccala, R., Hoebe, K., Kono, D. H., Beutler, B., & Theofilopoulos, A. N. (2007). TLR-dependent and TLR-independent pathways of type I interferon induction in systemic autoimmunity. Nature Medicine, 13 (5), 543-51. http://doi.org/10.1038/nm1590
21. Banerjee, S., Xie, N., Cui, H., Tan, Z., Yang, S., Icyuz, M., . . . Liu, G. (2013). MicroRNA let-7c regulates macrophage polarization. Journal of Immunology (Baltimore, Md.: 1950), 190 (12), 6542-9. http://doi.org/10.4049/jimmunol.1202496
22. Murray, P. J., Allen, J. E., Biswas, S. K., Fisher, E. A., Gilroy, D. W., Goerdt, S., . . . Wynn, T. A. (2014). Macrophage Activation and Polarization: Nomenclature and Experimental Guidelines. Immunity, 41 (1), 14-20. http://doi.org/10.1016/j.immuni.2014.06.008
23. Hao, N.-B., Lu, M.-H., Fan, Y.-H., Cao, Y.-L., Zhang, Z.-R., & Yang, S.-M. (2012). Macrophages in tumor microenvironments and the progression of tumors. Clinical & Developmental Immunology, 2012, 948098. http://doi.org/10.1155/2012/948098
24. Sinha, P., Clements, V. K., & Ostrand-Rosenberg, S. (2005). Reduction of myeloid-derived suppressor cells and induction of M1 macrophages facilitate the rejection of established metastatic disease. Journal of Immunology, 174 (2), 636-45. Retrieved from http://www.ncbi.nlm.nih.gov/pubmed/15634881
25. Bingle, L., Brown, N. J., & Lewis, C. E. (2002). The role of tumour-associated macrophages in tumour progression: implications for new anticancer therapies. The Journal of Pathology, 196 (3), 254-65. http://doi.org/10.1002/path.1027
26. Herbeuval, J.-P., Lambert, C., Sabido, O., Cottier, M., Fournel, P., Dy, M., & Genin, C. (2003). Macrophages from cancer patients: analysis of TRAIL, TRAIL receptors, and colon tumor. Journal of the National Cancer Institute, 95 (8), 611-21. Retrieved from http://www.ncbi.nlm.nih.gov/pubmed/12697854
27. Ma, J., Liu, L., Che, G., Yu, N., Dai, F., & You, Z. (2010). The M1 form of tumor-associated macrophages in non-small cell lung cancer is positively associated with survival time. BMC Cancer, 10, 112. http://doi.org/10.1186/1471-2407-10-112
28. Ohri, C. M., Shikotra, A., Green, R. H., Waller, D. a, & Bradding, P. (2009). Macrophages within NSCLC tumour islets are predominantly of a cytotoxic M1 phenotype associated with extended survival. The European Respiratory Journal, 33 (1), 118-26. http://doi.org/10.1183/09031936.00065708
29. Urban, J. L., Shepard, H. M., Rothstein, J. L., Sugarman, B. J., & Schreiber, H. (1986). Tumor necrosis factor: a potent effector molecule for tumor cell killing by activated macrophages. Proceedings of the National Academy of Sciences of the United States of America, 83 (14), 5233-7. Retrieved from http://www.pubmedcentral.nih.gov/articlerender.fcgi?artid=323925&tool=pmcentrez&rende rtype=abstract
30. Wong, S.-C., Puaux, A.-L., Chittezhath, M., Shalova, I., Kajiji, T. S., Wang, X., . . . Biswas, S. K. (2010). Macrophage polarization to a unique phenotype driven by B cells. European Journal of Immunology, 40 (8), 2296-307. http://doi.org/10.1002/eji.200940288
31. Hardison, S. E., Herrera, G., Young, M. L., Hole, C. R., Wozniak, K. L., & Wormley, F. L. (2012). Protective immunity against pulmonary cryptococcosis is associated with STAT1-mediated classical macrophage activation. Journal of Immunology (Baltimore, Md.: 1950), 189 (8), 4060-8. http://doi.org/10.4049/jimmunol.1103455
32. Wang, Y.-C., He, F., Feng, F., Liu, X.-W., Dong, G.-Y., Qin, H.-Y., . . . Han, H. (2010). Notch signaling determines the M1 versus M2 polarization of macrophages in antitumor immune responses. Cancer Research, 70 (12), 4840-9. http://doi.org/10.1158/0008-5472.CAN-10-0269
33. Cai, X., Yin, Y., Li, N., Zhu, D., Zhang, J., Zhang, C.-Y., & Zen, K. (2012). Re-polarization of tumor-associated macrophages to pro-inflammatory MI macrophages by microRNA-155. Journal of Molecular Cell Biology, 4 (5), 341-3. http://doi.org/10.1093/jmcb/mjs044
34. Wei, Y., Nazari-Jahantigh, M., Chan, L., Zhu, M., Heyll, K., Corbalán-Campos, J., . . . Schober, A. (2013). The microRNA-342-5p fosters inflammatory macrophage activation through an Akt1- and microRNA-155-dependent pathway during atherosclerosis. Circulation, 127 (15), 1609-19. http://doi.org/10.1161/CIRCULATIONAHA.112.000736
35. Squadrito, M. L., Etzrodt, M., De Palma, M., & Pittet, M. J. (2013). MicroRNA-mediated control of macrophages and its implications for cancer.Trends in Immunology, 34 (7), 350-9. http://doi.org/10.1016/j.it.2013.02.003
36. Biswas, S. K., Gangi, L., Paul, S., Schioppa, T., Saccani, A., Sironi, M., . . . Sica, A. (2006). A distinct and unique transcriptional program expressed by tumor-associated macrophages (defective NF-kappaB and enhanced IRF-3/STATI activation). Blood, 107 (5), 2112-22. http://doi.org/10.1182/blood-20May 1, 0428
37. Steidl, C., Lee, T., & Shah, S. (2010b). Tumor-associated macrophages and survival in classic Hodgkin's lymphoma. The New England Journal of Medicine, 362 (10), 875-885. Retrieved from http://www.nejm.org/doi/full/10.1056/NEJMoa0905680
38. Lin, E. Y., Li, J.-F., Gnatovskiy, L., Deng, Y., Zhu, L., Grzesik, D. a, . . . Pollard, J. W. (2006). Macrophages regulate the angiogenic switch in a mouse model of breast cancer. Cancer Research, 66 (23), 11238-46. http://doi.org/10.1158/0008-5472.CAN-06-1278
39. Hagemann, T., Wilson, J., Burke, F., Kulbe, H., Li, N. F., Plüddemann, A., . . . Balkwill, F. R. (2006). Ovarian cancer cells polarize macrophages toward a tumor-associated phenotype. The Journal of Immunology, 176 (8), 5023-32. Retrieved from http://www.ncbi.nlm.nih.gov/pubmed/16585599
40. Hagemann, T., Lawrence, T., McNeish, I., Charles, K. a, Kulbe, H., Thompson, R. G., . . . Balkwill, F. R. (2008). “Re-educating” tumor-associated macrophages by targeting NF-kappaB. The Journal of Experimental Medicine, 205 (6), 1261-8. http://doi.org/10.1084/jem.20080108
41. Mandal, P., Pratt, B. T., Barnes, M., McMullen, M. R., & Nagy, L. E. (2011). Molecular mechanism for adiponectin-dependent M2 macrophage polarization: link between the metabolic and innate immune activity of full-length adiponectin. The Journal of Biological Chemistry, 286 (15), 13460-9. http://doi.org/10.1074/jbc.M110.204644
42. Mantovani, A., Allavena, P., Sica, A., & Balkwill, F. (2008). Cancer-related inflammation. Nature, 454 (7203), 436-44. http://doi.org/10.1038/nature07205
43. Cortez-Retamozo, V., Etzrodt, M., Newton, A., Rauch, P. J., Chudnovskiy, A., Berger, C., . . . Pittet, M. J. (2012). Origins of tumor-associated macrophages and neutrophils. Proceedings of the National Academy of Sciences of the United States of America, 109 (7), 2491-6. http://doi.org/10.1073/pnas.1113744109
44. Hercus, T. R., Thomas, D., Guthridge, M. A., Ekert, P. G., King-Scott, J., Parker, M. W., & Lopez, A. F. (2009). The granulocyte-macrophage colony-stimulating factor receptor: linking its structure to cell signaling and its role in disease. Blood, 114 (7), 1289-98. http://doi.org/10.1182/blood-2008-12-164004
45. Smith, H. O., Stephens, N. D., Qualls, C. R., Fligelman, T., Wang, T., Lin, C.-Y., . . . Pollard, J. W. (2013). The clinical significance of inflammatory cytokines in primary cell culture in endometrial carcinoma. Molecular Oncology, 7 (1), 41-54. http://doi.org/10.1016/j.molonc.2012.07.002
46. West, R. B., Rubin, B. P., Miller, M. A., Subramanian, S., Kaygusuz, G., Montgomery, K., . . . van de Rijn, M. (2006). A landscape effect in tenosynovial giant-cell tumor from activation of CSFI expression by a translocation in a minority of tumor cells. Proceedings of the National Academy of Sciences of the United States of America, 103 (3), 690-5. http://doi.org/10.1073/pnas.0507321103
47. Lin, E. Y., & Pollard, J. W. (2007). Tumor-associated macrophages press the angiogenic switch in breast cancer. Cancer Research, 67 (11), 5064-6. http://doi.org/10.1158/0008-5472.CAN-07-0912
48. Dalton, H. J., Armaiz-Pena, G. N., Gonzalez-Villasana, V., Lopez-Berestein, G., Bar-Eli, M., & Sood, A. K. (2014). Monocyte subpopulations in angiogenesis. Cancer Research, 74 (5), 1287-93. http://doi.org/10.1158/0008-5472.CAN-13-2825
49. Saccani, A., Schioppa, T., Porta, C., Biswas, S. K., Nebuloni, M., Vago, L., . . . Sica, A. (2006). p50 nuclear factor-kappaB overexpression in tumor-associated macrophages inhibits M1inflammatory responses and antitumor resistance. Cancer Research, 66 (23), 11432-40. http://doi.org/10.1158/0008-5472.CAN-06-1867
50. Gazzaniga, S., Bravo, A. I., Guglielmotti, A., van Rooijen, N., Maschi, F., Vecchi, A., . . . Wainstok, R. (2007). Targeting tumor-associated macrophages and inhibition of MCP-1 reduce angiogenesis and tumor growth in a human melanoma xenograft. The Journal of Investigative Dermatology, 127 (8), 2031-41. http://doi.org/10.1038/sj.jid.5700827
51. Luo, Y., Zhou, H., & Krueger, J. (2006). Targeting tumor-associated macrophages as a novel strategy against breast cancer. Journal of Clinical Investigation, 116 (8), 2132-2141.http://doi.org/10.1172/JCI27648.2132
52. Zeisberger, S. M., Odermatt, B., Marty, C., Zehnder-Fjällman, a H. M., Ballmer-Hofer, K., & Schwendener, R. a. (2006). Clodronate-liposome-mediated depletion of tumour-associated macrophages: a new and highly effective antiangiogenic therapy approach. British Journal of Cancer, 95 (3), 272-81. http://doi.org/10.1038/sj.bjc.6603240
53. Bettencourt-Dias, M., Giet, R., Sinka, R., Mazumdar, a, Lock, W. G., Balloux, F., . . . Glover, D. M. (2004). Genome-wide survey of protein kinases required for cell cycle progression. Nature, 432 (7020), 980-7. http://doi.org/10.1038/nature03160
54. Geschwind, J. H., Vali, M., & Wahl, R. (2006). Effects of 3 bromopyruvate (hexokinase 2 inhibitor) on glucose uptake in lewis rats using 2-(F-18) fluoro-2-deoxy-d-glucose. In 2006 Gastrointestinal Cancers Symposium (pp. 12-14).
55. Wolf, A., Agnihotri, S., Micallef, J., Mukherjee, J., Sabha, N., Cairns, R., . . . Guha, A. (2011). Hexokinase 2 is a key mediator of acrobic glycolysis and promotes tumor growth in human glioblastoma multiforme. The Journal of Experimental Medicine, 208 (2), 313-26. http://doi.org/10.1084/jem.20101470
56. Blagih, J., & Jones, R. G. (2012). Polarizing macrophages through reprogramming of glucose metabolism. Cell Metabolism, 15 (6), 793-5. http://doi.org/10.1016/j.cmet.2012.05.008
57. Haschemi, A., Kosma, P., Gille, L., Evans, C. R., Burant, C. F., Starkl, P., . . . Wagner, O. (2012). The sedoheptulose kinase CARKL directs macrophage polarization through control of glucose metabolism. Cell Metabolism, 15 (6), 813-26. http://doi.org/10.1016/j.cmet.2012.04.023
58. Arranz, A., Doxaki, C., Vergadi, E., Martinez de la Torre, Y., Vaporidi, K., Lagoudaki, E. D., . . . Tsatsanis, C. (2012). Akt1 and Akt2 protein kinases differentially contribute to macrophage polarization. Proceedings of the National Academy of Sciences of the United States of America, 109 (24), 9517-22. http://doi.org/10.1073/pnas. 1119038109
59. Jones, R. G., & Thompson, C. B. (2007). Revving the engine: signal transduction fuels T cell activation. Immunity, 27 (2), 173-8. http://doi.org/10.1016/j.immuni.2007.07.008
60. Shu, C. J., Guo, S., Kim, Y. J., Shelly, S. M., Nijagal, A., Ray, P., . . . Witte, O. N. (2005). Visualization of a primary anti-tumor immune response by positron emission tomography. Proceedings of the National Academy of Sciences of the United States of America, 102 (48), 17412-7. http://doi.org/10.1073/pnas.0508698102
61. Van Ginderachter, J. A., Movahedi, K., Hassanzadeh Ghassabeh, G., Meerschaut, S., Beschin, A., Raes, G., & De Bactselier, P. (2006). Classical and alternative activation of mononuclear phagocytes: Picking the best of both worlds for tumor promotion. Immunobiology, 211 (6), 487-501. Retrieved from http://www.sciencedirect.com/science/article/pii/S0171298506000829
62. Mills, C. D., Shearer, J., Evans, R., & Caldwell, M. D. (1992). Macrophage arginine metabolism and the inhibition or stimulation of cancer. Journal of Immunology (Baltimore, Md.: 1950), 149 (8), 2709-14. Retrieved from http://www.ncbi.nlm.nih.gov/pubmed/1401910
63. Ji, Y., Sun, S., Xu, A., Bhargava, P., Yang, L., Lam, K. S. L., . . . Qi, L. (2012). Activation of natural killer T cells promotes M2 Macrophage polarization in adipose tissue and improves systemic glucose tolerance via interleukin-4 (IL-4)/STAT6 protein signaling axis in obesity. The Journal of Biological Chemistry, 287 (17), 13561-71. http://doi.org/10.1074/jbc.M112.350066
64. Andreesen, R., Scheibenbogen, C., & Brugger, W. (1990). Adoptive transfer of tumor cytotoxic macrophages generated in vitro from circulating blood monocytes: a new approach to cancer immunotherapy. Cancer Research, 7450-7456. Retrieved from http://cancerres.aacrjournals.org/content/50/23/7450.short
65. Korbelik, M., Naraparaju, V. R., & Yamamoto, N. (1997). Macrophage-directed immunotherapy as adjuvant to photodynamic therapy of cancer. British Journal of Cancer, 75 (2), 202-7. Retrieved from http://www.pubmedcentral.nih.gov/articlerender.fcgi?artid=2063270&tool=pmcentrez&r endertype=abstract
66. Ellem, K. A. O., Rourke, M. G. E. O., Johnson, G. R., Parry, G., Misko, I. S., Schmidt, C. W., . . . Mulligan, R. C. (1997). A case report: immune responses and clinical course of the first human use of granulocyte/macrophage-colony-stimulating-factor-transduced autologous melanoma. Cancer Immunology, Immunotherapy, 10-20. Retrieved from http://www.springerlink.com/index/JQ4EB21E4C7ADMT7.pdf
67. Gast, G. de, & Klümpen, H. (2000). immunotherapy with subcutaneous granulocyte macrophage colony-stimulating factor, low-dose interleukin 2, and interferon Î± in progressive metastatic melanoma. Clinical Cancer Research. Retrieved from http://clincancerres.aacrjournals.org/content/6/4/1267.short
68. Hill, H., Jr, T. C., & Sabel, M. (2002). Immunotherapy with Interleukin 12 and Granulocyte-Macrophage Colony-stimulating Factor-encapsulated Microspheres Coinduction of Innate and Adaptive Antitumor. Cancer Research. Retrieved from http://cancerres.aacrjournals.org/content/62/24/7254.short
69. Lokshin, A., Mayotte, J., & Levitt, M. (1995). Mechanism of Interferon Beta-Induced Squamous Differentiation and Programmed Cell Death in Human Non-Small-Cell Lung Cancer Cell Lines. Journal of the National Cancer Institute, 87, 206-212. Retrieved from http://jnci.oxfordjournals.org/content/87/3/206.short
70. Johns, T., & Mackay, I. (1992). Antiproliferative potencies of interferons on melanoma cell lines and xenografts: higher efficacy of interferon Î2. Journal of the National Cancer Institute, (type II), 1185-1190. Retrieved from http://jnci.oxfordjournals.org/content/84/15/1185
71. Qin, X.-Q., Runkel, L., Deck, C., DeDios, C., & Barsoum, J. (1997). Interferon-beta induces S phase accumulation selectively in human transformed cells. Journal of Interferon & Cytokine Research, 17 (6), 355-367. http://doi.org/10.1089/jir.1997.17.355
72. Zhang, F., Lu, W., & Dong, Z. (2002). Tumor-infiltrating macrophages are involved in suppressing growth and metastasis of human prostate cancer cells by INF-β gene therapy in nude mice. Clinical Cancer Research 2942-2951. Retrieved from http://clincancerres.aacrjournals.org/content/8/9/2942.short
73. Simpson, K. D., Templeton, D. J., & Cross, J. V. (2012). Macrophage Migration Inhibitory Factor Promotes Tumor Growth and Metastasis by Inducing Myeloid-Derived Suppressor Cells in the Tumor Microenvironment. The Journal of Immunology. http://doi.org/10.4049/jimmunol.1201161
74. Sanford, D. E., Belt, B. A., Panni, R. Z., Mayer, A., Deshpande, A. D., Carpenter, D., . . . Linchan, D. C. (2013). Inflammatory monocyte mobilization decreases patient survival in pancreatic cancer: a role for targeting the CCL2/CCR2 axis. Clinical Cancer Research: An Official Journal of the American Association for Cancer Research, 19 (13), 3404-15. http://doi.org/10.1158/1078-0432.CCR-13-0525
75. Schmall, A., Al-Tamari, H. M., Herold, S., Kampschulte, M., Weigert, A., Wietelmann, A., . . . Savai, R. (2014). Macrophage and Cancer Cell Crosstalk via CCR2 and CX3CR1 is a Fundamental Mechanism Driving Lung Cancer. American Journal of Respiratory and Critical Care Medicine. http://doi.org/10.1164/rccm.201406-11370C
76. Kimura, Y. N., Watari, K., Fotovati, A., Hosoi, F., Yasumoto, K., Izumi, H., . . . Ono, M. (2007). Inflammatory stimuli from macrophages and cancer cells synergistically promote tumor growth and angiogenesis. Cancer Science, 98 (12), 2009-18. http://doi.org/10.1111/j.1349-7006.2007.00633.x
77. Chen, H., Li, P., Yin, Y., Cai, X., Huang, Z., Chen, J., . . . Zhang, J. (2010). The promotion of type 1 T helper cell responses to cationic polymers in vivo via toll-like receptor-4 mediated IL-12 secretion. Biomaterials, 31 (32), 8172-80. http://doi.org/10.1016/j.biomaterials.2010.07.056
78. Rogers, T. L., & Holen, I. (2011). Tumour macrophages as potential targets of bisphosphonates. Journal of Translational Medicine, 9 (1), 177. http://doi.org/10.1186/1479-5876-9-177
79. Junankar, S., Shay, G., Jurczyluk, J., Ali, N., Down, J., Pocock, N., . . . Rogers, M. J. (2015). Real-time intravital imaging establishes tumor-associated macrophages as the extraskeletal target of bisphosphonate action in cancer. Cancer Discovery, 5 (1), 35-42. http://doi.org/10.1158/2159-8290.CD-14-0621
80. Huang, Z., Yang, Y., Jiang, Y., Shao, J., Sun, X., Chen, J., . . . Zhang, J. (2013). Anti-tumor immune responses of tumor-associated macrophages via toll-like receptor 4 triggered by cationic polymers. Biomaterials, 34 (3), 746-55. http://doi.org/10.1016/j.biomaterials.2012.09.062
81. Q. He, T. Fornander, H. Johansson et al., “Thymidine kinase 1 in serum predicts increased risk of distant or loco-regional recurrence following surgery in patients with early breast cancer,” Anticancer Research, vol. 26, no. 6, pp. 4753-4759, 2006.
82. K. L. O′Neill, M. Hoper, and G. W. Odling-Smee, “Can thymidine kinase levels in breast tumors predict disease recurrence?” Journal of the National Cancer Institute, vol. 84, no. 23, pp. 1825-1828, 1992.

What is claimed is:

1. A chimeric receptor, the chimeric receptor comprising
a cytoplasmic domain;
a transmembrane domain; and
an extracellular domain;
wherein the cytoplasmic domain comprises a cytoplasmic portion of a receptor that when activated polarizes a macrophage;
wherein a wild-type protein comprising the cytoplasmic portion does not comprise the extracellular domain.

2. The chimeric receptor of claim 1, wherein the binding of a ligand to the extracellular domain activates the cytoplasmic portion.

3. The chimeric receptor of claim 1, wherein the cytoplasmic portion is activated, it polarizes a macrophage to a M1 macrophage.

4. The chimeric receptor of claim 1, wherein the cytoplasmic portion is activated, it polarizes a macrophage to a M2 macrophage.

5. The chimeric receptor of claim 1, wherein the cytoplasmic portion comprises a cytoplasmic domain from a toll-like receptor, myeloid differentiation primary response protein (MYD88), toll-like receptor 3 (TLR3), toll-like receptor 4 (TLR4), toll-like receptor 7 (TLR7), toll-like receptor 8 (TLR8), toll-like receptor 9 (TLR9), myelin and lymphocyte protein (MAL), interleukin-1 receptor-associated kinase 1 (IRAK1), low affinity immunoglobulin gamma Fc region receptor III-A (FCGR3A), low affinity immunoglobulin gamma Fc region receptor II-a (FCGR2A), and high affinity immunoglobulin epsilon receptor subunit gamma (FCER1G).

6. The chimeric receptor of claim 2, where the ligand is selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

7. The chimeric receptor of claim 1, wherein the extracellular domain is an antibody or fragment thereof specific for a ligand selected from the group consisting of Thymidine Kinase (TK1), Hypoxanthine-Guanine Phosphoribosyltransferase (HPRT), Receptor Tyrosine Kinase-Like Orphan Receptor 1 (ROR1), Mucin-16 (MUC-16), Epidermal Growth Factor Receptor vIII (EGFRvIII), Mesothelin, Human Epidermal Growth Factor Receptor 2 (HER2), Carcinoembryonic Antigen (CEA), B-Cell Maturation Antigen (BCMA), Glypican 3 (GPC3), Fibroblast Activation Protein (FAP), Erythropoietin-Producing Hepatocellular Carcinoma A2 (EphA2), Natural Killer Group 2D (NKG2D) ligands, Disialoganglioside 2 (GD2), CD19, CD20, CD30, CD33, CD123, CD133, CD138, and CD171.

8. The chimeric receptor of claim 7, wherein the antibody or fragment thereof is a ScFv fragment.

9. The chimeric receptor of claim 1, wherein the chimeric receptor further comprises a linker between the transmembrane domain and the extracellular domain.

10. The chimeric receptor of claim 9, wherein the linker is a GS linker.

11. The chimeric receptor of claim 1, wherein the chimeric receptor further comprises a hinge region between the transmembrane domain and the extracellular domain.

12. The chimeric receptor of claim 9, wherein the chimeric receptor further comprises a hinge region between the transmembrane domain and the linker.

13. A nucleic acid comprising a polynucleotide encoding the chimeric receptor of claim 1.

14. The nucleic acid of claim 13, further comprising a promoter operably linked to the polynucleotide.

15. A vector comprising the nucleic acid of claim 13.

16. The vector according to claim 15, wherein the vector is a lentiviral vector.

17. A method of polarizing a macrophage, the method comprising:
contacting a macrophage comprising the chimeric receptor of claim 1 with a ligand for the extracellular domain of the chimeric receptor; and
binding the ligand to the extracellular domain of the chimeric receptor;
wherein the binding of the ligand to the extracellular domain of the chimeric receptor activates the cytoplasmic portion; and
wherein activation of the cytoplasmic portion polarizes the macrophage.
